(19) United States
(12) Patent Application Publication
(10) Pub. No.: US 2017/0183399 A1
(43) Pub. Date: Jun. 29, 2017

(54) IMMUNOPOTENTIATOR CONTAINING ANTI-ANG2 ANTIBODY

(71) Applicant: Samsung Electronics Co., Ltd., Suwon-si (KR)

(72) Inventors: Yuhoi Kang, Seongnam-si (KR); Hojun Lee, Seoul (KR); Hongseok Jo, Osan-si (KR); Sang Chul Park, Seongnam-si (KR); Sang Yeul Han, Yongin-si (KR)

(21) Appl. No.: 15/387,282

(22) Filed: Dec. 21, 2016

(30) Foreign Application Priority Data
Dec. 24, 2015 (KR) 10-2015-0186457

(51) Int. Cl. C07K 16/18

(52) U.S. Cl. CPC C07K 16/18; C07K 2317/565; C07K 2317/92; C07K 2317/24; C07K 2317/622

(57) ABSTRACT

Provided is a method of potentiating immunity or preventing or treating an immune-related disease comprising administering an anti-Ang2 antibody or an antigen-binding fragment thereof to a subject in need thereof.

### CROSS-REFERENCE TO RELATED APPLICATIONS

The present application claims priority to Korean Patent Application No. 10-2015-0186457 filed on Dec. 24, 2015, with the Korea Industrial Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 59,482 Byte ASCII (Text) file named “727051_ST25.TXT,” created on Dec. 21, 2016.

### BACKGROUND OF THE INVENTION

1. Field of the Invention Provided is a method of potentiating immunity or preventing and/or treating an immune-related disease comprising administering an anti-Ang2 antibody or an antigen-binding fragment thereof to a subject in need thereof.

2. Description of the Related Art

The attenuation of immune functions caused by senescence and various immune-related diseases (e.g., acquired immunodeficiency syndrome, autoimmune diseases, etc.) includes thymus involution, reduction in T cell proliferation, reduction in the response of T cells to foreign antigens, and an increase in immune response to autoantigens.

T cells are a type of immune cell that is involved in immunoregulation through the generation of cytokines responsible for immune cell proliferation and differentiation, and destruction of virus-infected cells, implanted cells, and tumor cells. T cells account for 60˜80% of peripheral blood lymphocytes, and can be distinguished from other lymphocytes by the presence of a T cell receptor (TCR) on the cell surface. The T cell receptor is responsible for recognizing fragments of antigens, and exists as a complex with CD2 and CD3. T cells are also divided into CD4+T cells and CD8+T cells according to the expression of CD4 or CD8. CD4+T cells help the activity of other immune cells by releasing T cell cytokines, and can activate macrophages, which mediate delayed hypersensitivity. CD8+T cells, known as cytotoxic T cells, mainly function to destroy virus-infected cells and tumor cells. CD8+T cells are also implicated in transplant rejection.

When T cells recognize an antigen, they actively proliferate and secrete immunostimulants such as IL-2 cytokine, which is a protein that incites the immune system to defend against the invasion of germs. IL-2, produced from activated T cells, acts through IL-2 receptors in an intrathymic autocrine/paracrine pathway. Signaling through IL-2 receptors activates the Akt, MAP kinase, and JAK-STAT pathway, regulating biological functions associated with cell differentiation, growth, and survival.

Recent reports have suggested that the down-regulation of c-myc, c-jun, and c-fos genes (i.e., genes that play an important role in T cell activation) and AP-1 and NF-AT (i.e., transcription regulators of IL-2 cytokine) play a role in senescence. Furthermore, during the progression of senescence or HIV (Human Immunodeficiency Virus) infection, IL-2 expression is suppressed, so that the immune system cannot exhibit an effective response.

Conventional immunity-enhancing methods using small molecules are restricted because they are prone to non-specific binding, which leads to side effects. In addition, the treatment of AIDS is generally conducted with Highly Active Anti-Retroviral Therapy (HAART), which can reduce the level of the virus. However, HAART does not have any immunopotentiation effects. Alternatively, the reduction of inflammation with hormones, such as glucocorticoids, have been used to prevent immunity attenuation. However, these methods are limited in terms of regimen and dosage because the drugs react with target molecules, causing side effects such as cardiovascular diseases, cancer, and osteoporosis.

Therefore there is a need for a therapy for alleviating the attenuation of immune function caused by senescence and/or immune-related diseases. This invention provides such a therapy.

### SUMMARY OF THE INVENTION

The present disclosure addresses a use of an anti-Ang2 antibody or an antigen-binding fragment thereof in immunopotentiation and/or in prevention and/or treatment of an immune-related disease.

An embodiment provides an immunopotentiator comprising an anti-Ang2 antibody or an antigen-binding fragment thereof.

Another embodiment provides a method of potentiating immunity, comprising administering an anti-Ang2 antibody or an antigen-binding fragment thereof to a subject in need of potentiating immunity.

A further embodiment provides a pharmaceutical composition for preventing or treating an immune-related disease, comprising an anti-Ang2 antibody or an antigen-binding fragment thereof and a pharmaceutically acceptable carrier.

Still another embodiment provides a method of preventing or treating an immune-related disease, comprising administering an anti-Ang2 antibody or an antigen-binding fragment thereof to a subject in need of preventing or treating an immune-related disease.

The anti-Ang2 antibody or antigen-binding fragment thereof useful in the present disclosure may be characterized in that it binds specifically to Ang2 (angiopoietin-2) without interfering with the interaction between Ang2 and Tie2, thereby inhibiting Ang2, and/or inducing the activation of the Tie2 receptor.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a photographic image showing a comparison in thymus size between subjects administered an anti-Ang2 antibody or a control antibody;

FIG. 2 is a graph in which the number of thymocytes is compared between subjects administered an anti-Ang2 antibody or a control antibody;

FIG. 3 is a graph in which the number of naive CD4+T cells is compared between subjects administered an anti-Ang2 antibody or a control antibody;

FIG. 4 is a graph in which the percentage of IL-2-secreting CD4+T cells is compared between subjects administered an anti-Ang2 antibody or a control antibody; and

FIG. 5 is a graph in which the percentage of self-renewal hematopoietic stem cells is compared between subjects administered an anti-Ang2 antibody or a control antibody.

### DETAILED DESCRIPTION

One embodiment provides a method for potentiating immunity, comprising administering an anti-Ang2 antibody or an antigen-binding fragment thereof to a subject in need thereof. The method may further comprise identifying (diagnosing or selecting) a subject that is in need of immunopotentiation prior to the administering step.

The immunopotentiation may refer to enhancement or increase in immunity, which may be exemplified by 1) improvement or increase in i) resistance to thymus involution, ii) thymic function, iii) the number of T cells, or iv) production of hematopoietic stem cells, or 2) augmentation or recovery of T cell functions, and the like.

A further embodiment of the present invention is a method for preventing thymus involution and/or improving a thymic function, comprising administering an anti-Ang2 antibody or an antigen-binding fragment thereof to a subject in need thereof. The method may further comprise identifying (diagnosing or selecting) a subject that is in need of preventing thymus involution and/or improving a thymic function prior to the administering step. As used herein, the term “thymus involution” is intended to encompass, but is not limited to, the reduction of thymus size, thymocyte count, and/or T cell (e.g., CD4+T cells) production function. Thymus involution may be caused by various factors comprising, but not limited to, senescence and/or immune diseases. The improvement of thymic functions means an improvement in thymus size, an increase in thymocyte count, and/or increased T cell production, but is not limited thereto.

Another embodiment is a method for augmenting T cells, comprising administering an anti-Ang2 antibody or an antigen-binding fragment thereof to a subject in need thereof. The method may further comprise identifying (diagnosing or selecting) a subject that is in need of augmenting T cells prior to the administering step. The T cells may be a type of CD4 positive (CD4+) T cells. Here, the augmentation of T cells means the increase of a T cell count and/or the amelioration of T cell function (e.g., a higher expression level of cytokines, such as IL-2, interferon-γ, TNF-β, IL-10, etc), but is not limited thereto. In particular, because a suitable population level of CD4+ immune cells is strongly correlated with high survival rates and favorable prognosis for immune diseases such as acquired immune deficiency syndrome, an increased count of CD4+T cells may be advantageous for immunopotentiation and treating various immune-related diseases, comprising acquired immune deficiency syndrome.

Another embodiment is a method for increasing the production of hematopoietic stem cells (self-renewal), comprising administering an anti-Ang2 antibody or an antigen-binding fragment thereof to a subject in need thereof. The method may further comprise identifying (diagnosing or selecting) a subject that is in need of increased production of hematopoietic stem cells (self-renewal) prior to the administering step. The hematopoietic stem cells may comprise, but are not limited to, self-renewing hematopoietic stem cells (e.g. long-term hematopoietic stem cells etc.).

Another embodiment provides a method for preventing or treating an immune-related disease, comprising administering an anti-Ang2 antibody or an antigen-binding fragment thereof to a subject in need thereof. The method may further comprise identifying (diagnosing or selecting) a subject that is in need of preventing or treating an immune-related disease, prior to the administering step. The immune-related disease may be at least one selected from the group consisting of acquired immune deficiency syndrome (AIDS) and autoimmune diseases (e.g., rheumatism, systemic lupus erythematosus, and autoimmune hemolytic anemia).

The anti-Ang2 antibody or the antigen-binding fragment thereof is an antibody or an antigen-binding fragment thereof that specifically binds to Ang2, but does not inhibit binding between Ang2 and a Tie2 receptor. The antibody, thus, can form a complex with a Tie2 receptor through Ang2 (antibody-Ang2-Tie2 complex). The antibody or the antigen-binding fragment is characterized by dimerization, through which the Tie2 receptors of the complexes can be effectively clustered, thus inducing the activation of the Tie2 receptor and its downstream signaling. In such a mechanism of action, the antibody binds to Ang2 to induce the internalization and degradation of Ang2, thereby inhibiting Ang2 and lowering the level of circulating Ang2. By conjugating a Tie2 receptor together with Ang2 the antibody or the antigen-binding fragment thereof activates the Tie2 receptor in an ‘Ang1-like’ manner to thus exhibit the dual function of inducing Tie2 downstream signaling and stabilizing vascular endothelial cells.

So long as it specifically recognize and binds to Ang2 and complexes with (coupled to) Tie2 through Ang2, any antibody or an antigen-binding fragment thereof may fall within the scope of the anti-Ang2 antibody or the antigen-binding fragment thereof in accordance with the present disclosure. Moreover, the anti-Ang2 antibody or the antigen-binding fragment thereof may induce the activation of a Tie2 receptor. Such Tie2 receptor activation may be triggered by phosphorylating a Tie2 receptor and/or a protein responsible for the downstream signal pathway thereof, for example, at least one protein selected from the group consisting Akt (NM_005163), eNOS (NM_000603), 42/44 (NM_002745), etc. Also, the anti-Ang2 antibody or the antigen-binding fragment thereof may induce the intracellular internalization of a Tie2 receptor. In other words, the anti-Ang2 antibody or the antigen-binding fragment thereof binds to Ang2 and induces the activation of a Tie2 receptor by forming a complex with a Tie2 receptor, together with Ang2, without inhibiting binding between Ang2 and the Tie2 receptor. An Ang2 protein, which acts as an antigen for the antibody provided by the present disclosure, is a soluble ligand present in the blood, functioning to promote tumor angiogenesis, metastasis, and invasion. Ang2 may be a protein originating from mammals comprising primates such as humans, monkeys, etc., and rodents such as mice, rats, etc. Examples of the Ang2 may comprise, but are not limited to, human Ang2 (e.g. NCBI Accession No. O15123), monkey Ang2 (e.g. NCBI Accession No. Q8MIK6), mouse Ang2 (e.g. NCBI Accession No. O35608), and rat Ang2 (e.g. NCBI Accession No. O35462).

The Tie2 receptor (TEK tyrosine kinase), which acts as an Angiopoietin-2 receptor, is expressed in vascular endothelial cells in various mammals such as mice (NM_013690; NP_038718), rats, and humans (NM_000459; NP_000450), and is involved in various downstream signaling pathways.

The anti-Ang2 antibody or the antigen-binding fragment thereof may recognize, as an epitope, full loop 1 (a region from 417^{th}to 434^{th} amino acid residue of SEQ ID NO: 11) of human Ang2 (hAng2; SEQ ID NO: 11; Accession # O15123) or a part thereof (e.g. at least one selected from the group consisting of externally exposed amino acid residues of the loop), or an amino acid sequence composed of 2 to 20, 2 to 15, 2 to 10, or 2 to 5 neighboring (consecutive) amino acids comprising at least one externally exposed amino acid residue of loop 1 of SEQ ID NO: 11, or may specifically bind thereto. As used herein, the term “externally exposed residue” refers to a residue that can be exposed to a biological medium (for example, a solution environment in vivo, e.g., physiological pH, temperature, isotonicity, etc.) to perform binding to a different protein.

Ang2
(SEQ ID NO: 11)
MWQIVFFTLS CDLVLAAAYN NFRKSMDSIG KKQYQVQHGS
CSYTFLLPEM DNCRSSSSPY VSNAVQRDAP LEYDDSVQRL
QVLENIMENN TQWLMKLENY IQDNMKKEMV EIQQNAVQNQ
TAVMIEIGTN LLNQTAEQTR KLTDVEAQVL NQTTRLELQL
LEHSLSTNKL EKQILDQTSE INKLQDKNSF LEKKVLAMED
KHIIQLQSIK EEKDQLQVLV SKQNSIIEEL EKKIVTATVN
NSVLQKQQHD LMETVNNLLT MMSTSNSAKD PTVAKEEQIS
FRDCAEVFKS GHTTNGIYTL TFPNSTEEIK AYCDMEAGGG
GWTIIQRRED GSVDFQRTWK EYKVGFGNPS GEYWLGNEFV
SQLTNQQRYV LKIHLKDWEG NEAYSLYEHF YLSSEELNYR
IHLKGLTGTA GKISSISQPG NDFSTKDGDN DKCICKCSQM
LTGGWWFDAC GPSNLNGMYY PQRQNTNKFN GIKWYYWKGS
GYSLKATTMM IRPADF

By way of example, the anti-Ang2 antibody may recognize and bind, as an epitope, Q418, P419, a combination of Q418 and P419 positioned at loop 1 of SEQ ID NO: 11, or a region composed of 2 to 20, 2 to 15, 2 to 10, or 2 to 5 neighboring (consecutive) amino acids comprising Q418, P419 or a combination of Q418 and P419 on SEQ ID NO: 11. In one embodiment, the anti-Ang2 antibody may recognize and specifically bind the amino acid residues Q418 and P419 on SEQ ID NO: 11 as an epitope.

Q418, P419, and an amino acid region comprising them, which is an epitope for the anti-Ang2 antibody, are exposed amino acid residues positioned at loop 1 of the three-dimensional structure of Ang2, and are not implicated in binding between Ang2 and a Tie2 receptor.

As used in the context of Q418, P419, or an amino acid region comprising them, to which the anti-Ang2 antibody binds, the term “neighboring” (consecutive) amino acids may refer to amino acids which are adjacent to one another on a primary, secondary, or tertiary protein structure.

The antibody may be humanized or affinity matured.

In some embodiments, the anti-Ang2 antibody or an antigen-binding fragment thereof may comprise:

a heavy-chain complementarity-determining region (CDR) comprising at least one selected from the group consisting of a polypeptide comprising the amino acid sequence of SEQ ID NO: 1 (CDR-H1), a polypeptide comprising the amino acid sequence of SEQ ID NO: 2 (CDR-H2), and a polypeptide comprising the amino acid sequence of SEQ ID NO: 3 (CDR-H3), or a heavy-chain variable region comprising the heavy-chain complementarity-determining region;

a light-chain complementarity-determining region selected from the group consisting of a polypeptide comprising the amino acid sequence of SEQ ID NO: 4 (CDR-L1), a polypeptide comprising the amino acid sequence of SEQ ID NO: 5 (CDR-L2), and a polypeptide comprising the amino acid sequence of SEQ ID NO: 6 (CDR-L3), or a light chain variable region comprising the light-chain complementarity-determining region;

a combination of the at least one heavy-chain complementarity-determining region and the at least one light-chain complementarity-determining region; or

a combination of the heavy-chain variable region and the light chain variable region.

According to a particular embodiment, the anti-Ang2 antibody or an antigen-binding fragment thereof may comprise, consist essentially of, or consist of:

a heavy-chain complementarity-determining region comprising a polypeptide comprising the amino acid sequence of SEQ ID NO: 1 (CDR-H1), a polypeptide comprising the amino acid sequence of SEQ ID NO: 2 (CDR-H2), and a polypeptide comprising the amino acid sequence of SEQ ID NO: 3 (CDR-H3), or a heavy-chain variable region comprising the at least one heavy-chain complementarity-determining region; and

a light-chain complementarity-determining region comprising a polypeptide comprising the amino acid sequence of SEQ ID NO: 4 (CDR-L1), a polypeptide comprising the amino acid sequence of SEQ ID NO: 5 (CDR-L2), and a polypeptide comprising the amino acid sequence of SEQ ID NO: 6 (CDR-L3), or a light chain variable region comprising the light-chain complementarity-determining region.

In one embodiment, the heavy-chain variable region of the anti-Ang2 antibody or the antigen-binding fragment may comprise, consist essentially of, or consist of the amino acid sequence of SEQ ID NO: 7:

(SEQ ID NO: 7)
DVQLQESGPDLVKPSQSLSLTCTVTGYSITSDYAWNWIRQFPGNKLEW
MGYINYSGNTDYNPSLKSRSSITRDTSKNQFFLQLNSVTTGDTATYYC
ARGNFEGAMDYWGQGTLVTVSS

(In SEQ ID NO: 7, the underlined bold letters represent CDR-H1, CDR-H2, and CDR-H3 in sequence)

The light chain variable region of the antibody according to one embodiment may comprise the amino acid sequence of SEQ ID NO: 9:

(SEQ ID NO: 9)
SIVMTQTPKFLLVSAGDRVTITCKASQSVSNDVAWYQQKPGQSPKLLI
YYASNRYPGVPDRFTGSGYGTDFTFTISTVQAEDLAVYFCQQDYSSPW
TFGGGTKLEIK

(In SEQ ID NO: 9, the underlined bold letters represent CDR-L 1, CDR-L2, and CDR-L3 in sequence)

Accordingly, the anti-Ang2 antibody or an antigen-binding fragment thereof may comprise, consist essentially of, or consist of a heavy-chain variable region comprising the amino acid sequence of SEQ ID NO: 7, a light chain variable region comprising the amino acid sequence of SEQ ID NO: 9, or a combination of the heavy-chain variable region and the light chain variable region.

For example, the anti-Ang2 antibody or an antigen-binding fragment thereof may comprise, consist essentially of, or consist of a heavy-chain variable region comprising the amino acid sequence of SEQ ID NO: 7, and a light chain variable region comprising the amino acid sequence of SEQ ID NO: 9.

In some embodiments, the anti-Ang2 antibody or an antigen-binding fragment thereof may be provided with enhanced affinity for Ang2 by partial substitution on the amino acid sequence of at least one CDR, for example, at least one selected from among CDR-H2, CDR-L1, and CFR-L3, while maintaining the intrinsic antibody activity.

According to an embodiment, the affinity-enhanced (affinity-matured) anti-Ang2 antibody or an antigen-binding fragment thereof may comprise at least one substitution selected from the group consisting of:

(1) substitution of the first amino acid residue Tyr (Y) on the amino acid sequence (YINYSGNTDYNPSLKS; SEQ ID NO: 2) of CDR-H2 with Lys (K);

(2) substitution of the third amino acid residue Asn (N) on the amino acid sequence (YINYSGNTDYNPSLKS; SEQ ID NO: 2) of CDR-H2 with Ser (S);

(3) substitution of the fifth amino acid residue Ser (S) on the amino acid sequence (YINYSGNTDYNPSLKS; SEQ ID NO: 2) of CDR-H2 with Ala (A);

(4) substitution of the seventh amino acid residue Asn (N) on the amino acid sequence (YINYSGNTDYNPSLKS; SEQ ID NO: 2) of CDR-H2 with Lys (K);

(5) substitution of the eleventh (last) amino acid residue Ala (A) on the amino acid sequence (KASQSVSNDVA; SEQ ID NO: 4) of CDR-L1 with His (H);

(6) substitution of the fifth amino acid residue Ser (S) on the amino acid sequence (KASQSVSNDVA; SEQ ID NO: 4) of CDR-L1 with Phe (F);

(7) substitution of the eighth amino acid residue Asn (N) on the amino acid sequence (KASQSVSNDVA; SEQ ID NO: 4) of CDR-L1 with Thr (T)

(8) substitution of the fourth amino acid residue Asn (N) on the amino acid sequence (YASNRYP; SEQ ID NO: 5) of CDR-L2 with Ile (I);

(9) substitution of the fifth amino acid residue Arg (R) on the amino acid sequence (YASNRYP; SEQ ID NO: 5) of CDR-L2 with Pro (P);

(10) substitution of the second amino acid residue Gln (Q) on the amino acid sequence (QQDYSSPWT; SEQ ID NO: 6) of CDR-L3 with His (H); and

(11) substitution of the eight amino acid residue Trp (W) on the amino acid sequence (QQDYSSPWT; SEQ ID NO: 6) of CDR-L3 with Phe (F).

In some embodiments, the affinity-enhanced anti-Ang2 antibody or an antigen-binding fragment thereof may comprise an amino acid sequence of Formula 1 (SEQ ID NO: 20), as a CDR-H2:

X1-I-X2-Y-X3-G-X4-T-D-Y-N-P-S-L-K-S (Formula 1: SEQ ID NO: 20)

wherein, X1 is Tyr (Y) or Lys (K), X2 is Asn (N) or Ser (S), X3 is Ser (S) or Ala (A), and X4 is Asn (N) or Lys (K).

According to another embodiment, the amino acid sequence of SEQ ID NO: 20 may be SEQ ID NO: 14 or SEQ ID NO: 15.

In some embodiments, the affinity-enhanced anti-Ang2 antibody and an antigen-binding fragment thereof may comprise an amino acid sequence of Formula 2 (SEQ ID NO: 21), as a CDR-L1:

K-A-S-Q-X5-V-S-X6-D-V-X7 (Formula 2: SEQ ID NO: 21)

wherein, X5 is Ser (S) or Phe (F), X6 is Asn (N) or Thr (T), and X7 is Ala (A) or His (H).

In a particular embodiment, the amino acid sequence of SEQ ID NO: 21 may be SEQ ID NO: 16 or SEQ ID NO: 17.

In some embodiments, the affinity-enhanced anti-Ang2 antibody and an antigen-binding fragment thereof may comprise an amino acid sequence of Formula 3 (SEQ ID NO: 22), as a CDR-L2:

Y-A-S-X8-X9-Y-P (Formula 3: SEQ ID NO: 22)

wherein, X8 is Asn (N) or Ile (I), and X9 is Arg (R) or Pro (P).

According to a particular embodiment, the amino acid sequence of SEQ ID NO: 22 may be SEQ ID NO: 18.

In some embodiments, the affinity-enhanced anti-Ang2 antibody and an antigen-binding fragment thereof may comprise an amino acid sequence of Formula 4 (SEQ ID NO: 23), as a CDR-L3:

Q-X10-D-Y-S-S-P-X11-T (Formula 4: SEQ ID NO: 23)

wherein, X10 is Gln (Q) or His (H), and X11 is Trp (W) or Phe (F).

According to a particular embodiment, the amino acid sequence of SEQ ID NO: 23 may be identical to that of SEQ ID NO: 19.

Accordingly, the affinity-enhanced anti-Ang2 antibody or an antigen-binding fragment thereof may comprise, consist essentially of, or consist of:

heavy-chain complementarity-determining regions (CDRs) comprising:

a polypeptide comprising the amino acid sequence of SEQ ID NO: 1 (CDR-H1),

a polypeptide comprising the amino acid sequence of SEQ ID NO: 2 (CDR-H2), and

a polypeptide comprising the amino acid sequence of SEQ ID NO: 3 (CDR-H3), or

a heavy-chain variable region comprising the heavy-chain complementarity-determining regions;

light-chain complementarity-determining regions comprising:

a polypeptide comprising the amino acid sequence of SEQ ID NO: 21 (CDR-L1),

a polypeptide comprising the amino acid sequence of SEQ ID NO: 22 (CDR-L2), and

a polypeptide comprising the amino acid sequence of SEQ ID NO: 23 (CDR-L3), or

a light chain variable region comprising the light-chain complementarity-determining regions;

a combination of the heavy-chain complementarity-determining regions and the light-chain complementarity-determining regions; or

a combination of the heavy-chain variable region and the light chain variable region.

In a particular embodiment, the affinity-enhanced anti-Ang2 antibody or an antigen-binding fragment thereof may comprise:

heavy-chain complementarity-determining regions comprising:

a polypeptide comprising the amino acid sequence of SEQ ID NO: 1 (CDR-H1),

a polypeptide comprising an amino acid sequence selected from the group consisting of SEQ ID NO: 2, SEQ ID NO: 14, and SEQ ID NO: 15 (CDR-H2), and

a polypeptide comprising the amino acid sequence of SEQ ID NO: 3 (CDR-H3), or

a heavy-chain variable region comprising the heavy-chain complementarity-determining regions;

light-chain complementarity-determining regions comprising:

a polypeptide comprising the amino acid sequence of SEQ ID NO: 4, SEQ ID NO: 16, or SEQ ID NO: 17 (CDR-L1),

a polypeptide comprising the amino acid sequence of SEQ ID NO: 5 or SEQ ID NO: 18 (CDR-L2), and

a polypeptide comprising the amino acid sequence of SEQ ID NO: 6 or SEQ ID NO: 19, or

a light chain variable region comprising the light-chain complementarity-determining regions;

a combination of the heavy-chain complementarity-determining regions and the light-chain complementarity-determining regions; or

a combination of the heavy-chain variable region and the light-chain variable region.

In an embodiment, for the purpose of being distinguished from the template anti-Ang2-antibody or antibody fragment, the affinity-enhanced anti-Ang2 antibody or an antigen-binding fragment does not comprise all of a CDR-H1 comprising the amino acid sequence of SEQ ID NO: 1, a CDR-H2 comprising the amino acid sequence of SEQ ID NO: 2, a CDR-H3 comprising the amino acid sequence of SEQ ID NO: 3, a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6.

The heavy-chain complementarity-determining regions and light-chain complementarity-determining regions of the above-described templates and affinity-enhanced anti-Ang2 antibodies or their antigen-binding fragments are summarized in Table 1.

TABLE 1
Amino acid sequence of heavy chain CDR

| | CDRH1- | CDRH2- | CDRH3- |
|---|---|---|---|
| | KABAT | KABAT | KABAT |
| Template | SDYAWN | YINYSGNTDYNPSLKS | GNFEGAMDY |
| | (SEQ ID | (SEQ ID NO: 2) | (SEQ ID |
| | NO: 1) | | NO: 3) |
| Affinity- | — | KISYSGKTDYNPSLKS | — |
| matured | | (SEQ ID NO: 14) | |
| | — | KINYAGNTDYNPSLKS | — |
| | | (SEQ ID NO: 15) | |

Amino acid sequence of light chain CDR

| | CDRL1- | CDRL2- | CDRL3- |
|---|---|---|---|
| | KABAT | KABAT | KABAT |
| Template | KASQSVSNDVA | YASNRYP | QQDYSSPWT |
| | (SEQ ID | (SEQ ID NO: 5) | (SEQ ID |
| | NO: 4) | | NO: 6) |
| Affinity- | KASQSVSNDVH | YASIPYP | QHDYSSPFT |
| matured | (SEQ ID | (SEQ ID NO: 18) | (SEQ ID |
| | NO: 16) | | NO: 19) |
| | KASQFVSTDVH | | |
| | (SEQ ID | | |
| | NO: 17) | | |

In some embodiments, the affinity-enhanced anti-Ang2 antibody or an antigen-binding fragment thereof may be selected from the group consisting of:

(a) an antibody or an antigen-binding fragment thereof comprising:

a heavy-chain complementarity-determining region comprising the CDR-H1 of SEQ ID NO: 1, the CDR-H2 of SEQ ID NO: 14, and the CDR-H3 of SEQ ID NO: 3, or a heavy-chain variable region comprising the heavy-chain complementarity-determining region; and

a light-chain complementarity-determining region comprising the CDR-L1 of SEQ ID NO: 4, the CDR-L2 of SEQ ID NO: 5, the CDR-L3 of SEQ ID NO: 6, or a light chain variable region comprising the light-chain complementarity-determining region;

(b) an antibody or an antigen-binding fragment thereof comprising:

a heavy-chain complementarity-determining region comprising the CDR-H1 of SEQ ID NO: 1, the CDR-H2 of SEQ ID NO: 14, and the CDR-H3 of SEQ ID NO: 3, or a heavy-chain variable region comprising the heavy-chain complementarity-determining region; and

a light-chain complementarity-determining region comprising the CDR-L1 of SEQ ID NO: 16, the CDR-L2 of SEQ ID NO: 5, and the CDR-L3 of SEQ ID NO: 6; or a light chain variable region comprising the light-chain complementarity-determining region;

(c) an antibody or an antigen-binding fragment thereof comprising:

a heavy-chain complementarity-determining region comprising the CDR-H1 of SEQ ID NO: 1, the CDR-H2 of SEQ ID NO: 15, and the CDR-H3 of SEQ ID NO: 3, or a heavy-chain variable region comprising the heavy-chain complementarity-determining region; and

a light-chain complementarity-determining region comprising the CDR-L1 of SEQ ID NO: 4, the CDR-L2 of SEQ ID NO: 5, and the CDR-L3 of SEQ ID NO: 6, or a light chain variable region comprising the light-chain complementarity-determining region;

(d) an antibody or an antigen-binding fragment thereof comprising:

a heavy-chain complementarity-determining region comprising the CDR-H1 of SEQ ID NO: 1, the CDR-H2 of SEQ ID NO: 15, and the CDR-H3 of SEQ ID NO: 3, or a heavy-chain variable region comprising the heavy-chain complementarity-determining region; and

a light-chain complementarity-determining region comprising the CDR-L1 of SEQ ID NO: 16, the CDR-L2 of SEQ ID NO: 5, and the CDR-L3 of SEQ ID NO: 6, or a light chain variable region comprising the light-chain complementarity-determining region;

(e) an antibody or an antigen-binding fragment thereof comprising:

a heavy-chain complementarity-determining region comprising the CDR-H1 of SEQ ID NO: 1, the CDR-H2 of SEQ ID NO: 2, and the CDR-H3 of SEQ ID NO: 3, or heavy-chain variable region comprising the heavy-chain complementarity-determining region; and

a light-chain complementarity-determining region comprising the CDRL1 of SEQ ID NO: 16, the CDR-L2 of SEQ ID NO: 5, and the CDR-L3 of SEQ ID NO: 19, or a light chain variable region comprising the light-chain complementarity-determining region;

(f) an antibody or an antigen-binding fragment thereof comprising:

a heavy-chain complementarity-determining region comprising the CDR-H1 of SEQ ID NO: 1, the CDR-H2 of SEQ ID NO: 14, and the CDR-H3 of SEQ ID NO: 3, or a heavy-chain variable region comprising the heavy-chain complementarity-determining region; and

a light-chain complementarity-determining region comprising the CDRL1 of SEQ ID NO: 17, the CDR-L2 of SEQ ID NO: 5, and the CDR-L3 of SEQ ID NO: 6, or a light chain variable region comprising the light-chain complementarity-determining region;

(g) an antibody or an antigen-binding fragment thereof comprising:

a heavy-chain complementarity-determining region comprising the CDR-H1 of SEQ ID NO: 1, the CDR-H2 of SEQ ID NO: 14, and the CDR-H3 of SEQ ID NO: 3, or a heavy-chain variable region comprising the heavy-chain complementarity-determining region; and

a light-chain complementarity-determining region comprising the CDR-L1 of SEQ ID NO: 4, the CDR-L2 of SEQ ID NO: 18, and the CDR-L3 of SEQ ID NO: 6, or a light chain variable region comprising the light-chain complementarity-determining region;

(h) an antibody or an antigen-binding fragment thereof comprising:

a heavy-chain complementarity-determining region comprising the CDR-H1 of SEQ ID NO: 1, the CDR-H2 of SEQ ID NO: 14, and the CDR-H3 of SEQ ID NO: 3, or a heavy-chain variable region comprising the heavy-chain complementarity-determining region; and

a light-chain complementarity-determining region comprising the CDR-L1 of SEQ ID NO: 16, the CDR-L2 of SEQ ID NO: 18, and the CDR-L3 of SEQ ID NO: 6, or a light chain variable region comprising the light-chain complementarity-determining region; and

(i) an antibody or an antigen-binding fragment thereof comprising:

a heavy-chain complementarity-determining region comprising the CDR-H1 of SEQ ID NO: 1, the CDR-H2 of SEQ ID NO: 14, and the CDR-H3 of SEQ ID NO: 3, or a heavy-chain variable region comprising the heavy-chain complementarity-determining region; and

a light-chain complementarity-determining region comprising the CDR-L1 of SEQ ID NO: 17, the CDR-L2 of SEQ ID NO: 18, and the CDR-L3 of SEQ ID NO: 6, or a light chain variable region comprising the light-chain complementarity-determining region.

The affinity-enhanced anti-Ang2 antibody or an antigen-binding fragment thereof may have an affinity (KD) of 10 nM or less, 5 nM or less, 2 nM or less, or 1 nM or less, for example, 0.01 to 10 nM, 0.01 to 5 nM, 0.01 to 2 nM, or 0.01 to 1 nM for Ang2. This affinity is significantly increased compared to the template anti-Ang2 antibody, showing an affinity (KD) of about 8 nM for Ang2.

Another embodiment addresses a humanized anti-Ang2 antibody or an antigen-binding fragment thereof. The humanized anti-Ang2 antibody or antigen-binding fragment thereof can be constructed by substituting some of the amino acid residues on the framework other than the complementarity-determining region of the heavy-chain variable region (SEQ ID NO: 7). The amino acid sequences of the heavy-chain framework available for the construction of such a humanized anti-Ang2 antibody or antigen-binding fragment thereof are listed in Table 2, below.

TABLE 2
(Humanization of heavy chain)

| | FR1 (framework | FR2 (frame- | FR3 (frame- | FR4 (frame- |
|---|---|---|---|---|
| | adjacent to N- | work between | work between | work adjacent |
| | terminus of | CDR-H1 and | CDR-H2 and | to C-terminus |
| | CDR-H1) | CDR-H2) | CDR-H3) | of CDR-H3) |
| Frameworks in | DVQLQESGPDLV | WIRQFPGNKLEW | RSSITRDTSKNQF | WGQGTLVTVSS |
| the heavy chain | KPSQSLSLTCTVT | MG(SEQ ID | FLQLNSVTTGDT | (SEQ ID |
| variable region | GYSIT(SEQ ID | NO: 29) | ATYYCAR(SEQ | NO: 39) |
| of the template | NO: 24) | | ID NO: 34) | |
| (SEQ ID NO: 7) | | | | |
| Frameworks in | QVQLQESGPGLV | WIRQPPGKGLEW | RVTISVDTSKNQ | WGQGTLVTVSS |
| A heavy chain | KPSETLSLTCAVS | IG(SEQ ID | FSLKLSSVTAAD | (SEQ ID |
| variable region | GYSIS(SEQ ID | NO: 30) | TAVYYCAR(SEQ | NO: 40) |
| of a humanized | NO: 25) | | ID NO: 35) | |
| antibody (VH- | | | | |
| hu1) | | | | |
| Frameworks in | QVQLQESGPGLV | WIRQPPGKGLEW | RSTISRDTSKNQF | WGQGTLVTVSS |
| a heavy chain | KPSETLSLTCAVS | MG(SEQ ID | SLKLSSVTAADT | (SEQ ID |
| variable region | GYSIT(SEQ ID | NO: 31) | AVYYCAR(SEQ | NO: 41) |
| of a humanized | NO: 26) | | ID NO: 36) | |
| antibody (VH- | | | | |
| hu2) | | | | |
| Frameworks in | QVQLQESGPGLV | WIRQPPGKGLEW | RVTISVDTSKNQ | WGQGTLVTVSS |
| a heavy chain | KPSETLSLTCAVS | IG(SEQ ID | FSLKLSSVTAAD | (SEQ ID |
| variable region | GYSIT(SEQ ID | NO: 32) | TAVYYCAR(SEQ | NO: 42) |
| of a humanized | NO: 27) | | ID NO: 37) | |
| antibody (VH- | | | | |
| hu5) | | | | |
| Frameworks in | EVQLVESGGGLV | WVRQAPGKGLE | RSTISRDTSKNTF | WGQGTLVTVSS |
| a heavy chain | QPGGSLRLSCAA | WMG(SEQ ID | YLQMNSLRAED | (SEQ ID |
| variable region | SGYSIT(SEQ ID | NO: 33) | TAVYYCAR(SEQ | NO: 43) |
| of a humanized | NO: 28) | | ID NO: 38) | |
| antibody (VH- | | | | |
| hu3) | | | | |

In addition, the humanized anti-Ang2 antibody or antigen-binding fragment thereof can be constructed by substituting some of the amino acid residues on the framework other than the complementarity-determining region of the light-chain variable region (SEQ ID NO: 9). Amino acid sequences of the light-chain framework available for the construction of the humanized anti-Ang2 antibody or an antigen-binding fragment thereof are listed in Table 3, below.

TABLE 3
(Humanization of light chain)

| | FR1 (frame- | FR2 (frame- | FR3 (frame- | FR4 (frame- |
|---|---|---|---|---|
| | work adjacent | work between | work between | work adjacent |
| | to N-terminus | CDR-L1 and | CDR-L2 and | to C-terminus |
| | of CDR-L1) | CDR-L2) | CDR-L3) | of CDR-L3) |
| Frameworks | SIVMTQTPKFLLV | WYQQKPGQSPKL | GVPDRFTGSGYG | FGGGTKLEIK |
| in the light | SAGDRVTITC | LIY(SEQ ID | TDFTFTISTVQAE | (SEQ ID |
| chain variable | (SEQ ID | NO: 46) | DLAVYFC(SEQ | NO: 50) |
| region of the | NO: 44) | | ID NO: 48) | |
| template (SEQ | | | | |
| ID NO: 9) | | | | |
| Frameworks | DIQMTQSPSSLSA | WYQQKPGKAPKL | GVPSRFSGSGSGT | FGQGTKVEIK |
| in a light | SVGDRVTITC | LIY(SEQ ID | DFTLTISSLQPEDF | (SEQ ID |
| chain variable | (SEQ ID | NO: 47) | ATYYC(SEQ | NO: 51) |
| region of a | NO: 45) | | ID NO: 49) | |
| humanized | | | | |
| antibody (VL- | | | | |
| hu1) | | | | |

In accordance with a specific embodiment thereof, the present disclosure addresses a humanized anti-Ang2 antibody comprising a heavy-chain variable region, a light-chain variable region, or both thereof, or an antigen-binding fragment thereof, wherein

the heavy-chain variable region comprises:

a polypeptide comprising an amino acid sequence selected from the group consisting of SEQ ID NO: 24, SEQ ID NO: 25, SEQ ID NO: 26, SEQ ID NO: 27, or SEQ ID NO: 28, for example, SEQ ID NO: 25 to SEQ ID NO: 28, as an N-terminal framework region of CDR-H1 (framework adjacent to N-terminus of CDR-H1);

a polypeptide comprising an amino acid sequence selected from the group consisting of SEQ ID NO: 29, SEQ ID NO: 30, SEQ ID NO: 31, SEQ ID NO: 32, or SEQ ID NO: 33. For example, SEQ ID NO: 30 to SEQ ID NO: 33, as a framework region between CDR-H1 and CDR-H2;

a polypeptide comprising an amino acid sequence selected from the group consisting of SEQ ID NO: 34, SEQ ID NO: 35, SEQ ID NO: 36, SEQ ID NO: 37, or SEQ ID NO: 38. For example, SEQ ID NO: 35 to SEQ ID NO: 38, as a framework region between CDR-H2 and CDR-H3, and

a polypeptide comprising an amino acid sequence selected from the group consisting of SEQ ID NO: 39, SEQ ID NO: 40, SEQ ID NO: 41, SEQ ID NO: 42, or

SEQ ID NO: 43, for example, SEQ ID NO: 40 to SEQ ID NO: 43, as a C-terminal framework region of CDR-H3 (framework adjacent to C-terminus of CDR-H3); and

the light-chain variable region comprises:

a polypeptide comprising the amino acid sequence of SEQ ID NO: 44 or SEQ ID NO: 45, for example, the amino acid sequence of SEQ ID NO: 45, as an N-terminal framework region of CDR-L1 (framework adjacent to N-terminus of CDR-L1),

a polypeptide comprising the amino acid sequence of SEQ ID NO: 46 or SEQ ID NO: 47, for example, the amino acid sequence of SEQ ID NO: 47, as a framework region between CDR-L1 and CDR-L2,

a polypeptide comprising the amino acid sequence of SEQ ID NO: 48 or SEQ ID NO: 49, for example, the amino acid of SEQ ID NO: 49, as a framework region between CDR-L2 and CDR-L3, and

a polypeptide comprising the amino acid sequence of SEQ ID NO: 50 or SEQ ID NO: 51, for example, the amino acid sequence of SEQ ID NO: 51, as a C-terminal framework region of CDR-L3 (framework adjacent to C-terminus of CDR-L3).

In an embodiment, for the purpose of being distinguished from the template anti-Ang2 antibody or antigen-binding fragment thereof, the humanized anti-Ang2 antibody or an antigen-binding fragment thereof is not an antibody or antibody fragment in which the heavy-chain variable region comprises the amino acid sequence of SEQ ID NO: 24 as an N-terminal framework region of CDR-H1, the amino acid sequence of SEQ ID NO: 29 as a framework region between CDR-H1 and CDR-H2, the amino acid sequence of SEQ ID NO: 34 as a framework region between CDR-H2 and CDR-H3, and the amino acid sequence of CDR-H3 as a C-terminal framework region; and the light-chain variable region comprises the amino acid sequence of SEQ ID NO: 44 as an N-terminal framework region of CDR-L1, the amino acid sequence of SEQ ID NO: 46 as a framework region between CDR-L1 and CDR-L2, the amino acid sequence of SEQ ID NO: 48 as a framework region between CDR-L2 and CDR-L3, and the amino acid sequence of SEQ ID NO: 50 as a C-terminal framework region of CDR-L3.

In a particular embodiment, the humanized anti-Ang2 antibody or antigen-binding fragment thereof may comprise a heavy-chain variable region comprising an amino acid sequence selected from the group consisting of SEQ ID NO: 52, SEQ ID NO: 53, SEQ ID NO: 54, SEQ ID NO: 55, and SEQ ID NO: 56, and a light-chain variable region comprising an amino acid sequence selected from the group consisting of SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 87, and SEQ ID NO: 89.

The anti-Ang2 antibody useful in the present disclosure may be an anti-Ang2 antibody produced by the hybridoma cell line of Accession No. KCLRF-BP-00295, or may be an affinity-enhanced and/or humanized antibody derived therefrom.

Also, all the anti-Ang2 antibodies or their antigen-binding fragments described in Korean Patent Unexamined Application Publication No. 2015-0136031, which is hereby incorporated in its entirety by reference, fall within the scope of the present disclosure.

An animal-derived antibody that is produced by immunizing an animal with a desired antigen may generally trigger an immune rejection response when administered to humans for treatment purposes, and thus a chimeric antibody has been developed to suppress such immune rejection response. A chimeric antibody is formed by replacing the constant region of an animal-derived antibody, which is the cause of such an anti-isotype response, with that of a human antibody, using a genetic engineering method. Although the chimeric antibody is considerably mitigated in anti-isotype response in comparison with animal-derived antibodies, animal-derived amino acids present in the variable regions still retain the potential for side effects resulting from an anti-idiotypic response. Therefore, a humanized antibody has been developed to further mitigate such side effects. This can be constructed by grafting CDR (complementarity determining regions), which, of the variable regions of a chimeric antibody, play an important role in antigen binding, into a human antibody framework.

According to one embodiment, the antibody may be an animal-derived antibody (e.g., a mouse-derived antibody, etc.), a chimeric antibody (e.g., a mouse-human chimeric antibody, etc.), a humanized antibody, or a human antibody. The antibody or antigen-binding fragment thereof may be isolated from a living body or may be a non-naturally occurring substance. In this case, the antibody or antigen-binding fragment thereof may be recombinant or synthetic.

An intact antibody has a structure composed of two full-length light chains and two full-length heavy chains, with linkages between the light chains and the heavy chains via disulfide bonds. The constant region of an antibody is divided into a heavy chain constant region and a light chain constant region. The heavy chain constant region has gamma (γ), mu (μ), alpha (α), delta (δ) and epsilon (ε) types, and has gamma1 (γ1), gamma2 (γ2), gamma3 (γ3), gamma4 (γ4), alpha1 (α1) and alpha2 (α2) as its subclasses. The light chain constant region has kappa (κ) and lambda (λ) types.

As used herein, the term “heavy chain” is intended to encompass a full-length heavy chain, which consists of a variable region domain V_{H} comprising an amino acid sequence having variable region sequences sufficient to provide specificity for antigen binding, three constant region domains C_{H1}, C_{H2} and C_{H3} domains, and a hinge, and a fragment thereof. The term “light chain” is understood to encompass a full-length light chain, which consists of a variable region domain V_{L}, comprising an amino acid sequence having variable region sequences sufficient to contribute to specificity for antigen binding, a constant region domain C_{L}, and a fragment thereof.

The term “CDR (Complementarity Determining Region)” refers to an amino acid sequence found in the hypervariable region of a heavy chain and a light chain of an immunoglobulin. The heavy and light chain each comprise three CDRs (CDRH1, CDRH2, CDRH3, and CDRL1, CDRL2, CDRL3). The CDRs of an antibody can provide an essential contact residue for binding to an antigen or an epitope.

Throughout the specification, the terms “specifically binding” or “specifically recognizing” has the same meaning as generally known to a person of ordinary skill in the art, indicating that an antigen and an antibody specifically interact with each other to cause an immunological response.

The antigen-binding fragment of an antibody, provided by the present disclosure, may be a fragment comprising at least one complementarity determining region.

The term “antigen-binding fragment,” means a fragment of the full structure of an immunoglobulin, which is a partial polypeptide comprising a domain to which an antigen can bind. For example, it may be scFv, (scFv)₂, scFv-Fc, Fab, Fab′, or F(ab′)₂, but is not limited thereto.

Of the antigen-binding fragments, a Fab fragment is characterized by a structure composed of one variable and one constant domain from the light chain, and one variable and the first constant (C_{H1}) domain from the heavy chain, retaining one paratope. A Fab′ fragment is different from Fab in that Fab′ further comprises a hinge region having at least one cysteine residue at the C-terminus of the heavy chain C_{H1} domain. A F(ab′)₂ fragment forms as two Fab′ fragments are joined by a disulfide bond between the cysteine residues of the hinge region. An Fv fragment is a minimal antibody fragment, having only heavy chain variable regions and light chain variable regions, and recombinant techniques for producing the Fv fragment are well known in the art. In a two-chain Fv fragment, the heavy chain variable domains are associated with the light chain variable domains via a non-covalent bond. A single-chain Fv fragment has a structure in which a heavy chain variable domain and a light chain variable domain are covalently joined to each other, either via a covalent bond or directly at the C-terminus, so that it can form a dimer, as in a two-chain Fv fragment. In this context, the heavy chain variable region and the light chain variable region may be connected with each other, either through a linker such as a peptide linker or directly. The peptide linker may be composed of 1 to 100 amino acid residues or, more preferably, 2 to 50 amino acid residues. Amino acid sequences suitable for use in the peptide linker may be those well known in the art. The antigen-binding fragments may be produced using proteases (for example, a complete antibody can be restrictedly digested into Fab with papain and into F(ab′)₂ with pepsin) or a recombinant DNA technique.

Except for its CDR or variable regions, the anti-Ang2 antibody or an antigen-binding fragment thereof may be derived from the constant region of a human antibody. For example, the constant region of the antib-Ang2 antibody can be derived from IgA, IgE, or IgG, e.g., IgG1, IgG2, IgG 3, or IgG4.

The anti-Ang2 antibody may be monoclonal or polyclonal. Monoclonal antibodies may be prepared using a method known in the art, for example, a phage display technique. Alternately, the anti-Ang2 antibody may be prepared into a mouse-derived monoclonal antibody by methods set forth in Schwaber et al. (Schwaber, J and Cohen, E. P., “Human x Mouse Somatic Cell Hybrid Clones Secreting Immunoglobulins of Both Parental Types,” Nature, 244 (1973), 444-447).

Meanwhile, individual monoclonal antibodies may be screened using a typical ELISA (Enzyme-Linked ImmunoSorbent Assay) format, based on the binding potential with Ang2. Inhibitory activity can be verified through functional analysis such as competitive ELISA for verifying the molecular interaction of binding assemblies or functional analysis such as a cell-based assay. Then, monoclonal antibody members selected on the basis of their strong inhibitory activities may each be verified for their affinities (K_{d} values) to Ang2. In the method, the subject to which the antibody is administered may be a mammal, examples of which comprise primates such as humans or a monkeys, and rodents such as rats and mice, or may be a biological sample isolated from mammals or artificially cultured, such as cells, tissues, body fluids, etc.

The anti-Ang2 antibody or an antigen-binding fragment thereof may be used as an active ingredient in a pharmaceutical composition. The pharmaceutical composition may comprise a pharmaceutically acceptable carrier. Examples of the pharmaceutically acceptable carrier available for the pharmaceutical composition of the present disclosure comprise lactose, dextrose, sucrose, sorbitol, mannitol, starch, gum acacia, calcium phosphate, alginate, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and mineral oil, but are not limited thereto. Further, the pharmaceutical composition may comprise a typical additive selected from the group consisting of a diluent, an excipient, a lubricant, a humectant, a sweetener, a flavor enhancer, an emulsifier, a suspending agent, a preservative, and a combination thereof.

The pharmaceutical composition, or the antibody or its antigen-binding fragment, may be administered orally or parenterally. Parenteral administration may be carried out via intravenous, subcutaneous, intramuscular, intraperitoneal, intradermal, local, intranasal, intrapulmonary, and intrarectal routes. For oral administration, however, the pharmaceutical composition is preferably coated or formulated to protect the active ingredient from being degraded in the stomach because proteins or peptides are digested by pepsin. In addition, the administration may be performed with the aid of an instrument adapted for delivering the pharmaceutical composition to target cells.

The pharmaceutical composition, or the antibody or its antigen-binding fragment, may be administered in a pharmaceutically effective amount. The term “pharmaceutically effective amount”, as used herein, refers to an amount at which the active ingredient can exert a desired effect. The pharmaceutically effective amount may be determined in consideration of various factors comprising the type of formulation, the type of administration, the patient's age, weight, and sex, the severity of the disorder being treated, diet, the time and interval of administration, the route of administration, the rate of excretion, and sensitivity.

The content of the anti-Ang2 antibody or its antigen-binding fragment in the pharmaceutical composition may vary depending on various factors comprising the type of formulation, the type of administration, the patient's age, weight, and sex, the severity of the disorder being treated, diet, the time and interval of administration, the route of administration, the rate of excretion, and sensitivity. For example, the daily dose of the anti-Ang2 antibody or an antigen-binding fragment thereof may be on the order of 0.001 to 1000 mg/kg, particularly on the order of 0.01 to 100 mg/kg, and more particularly on the order of 0.1 to 50 mg/kg, but is not limited thereto. A daily dose may be formulated into a unit dose form or distributed into separate dose forms, or may be comprised within a multiple dose package.

The pharmaceutical composition may be formulated into a solution in an oily or aqueous medium, a suspension, a syrup, an emulsion, an elixir, a powder, a granule, a tablet, or a capsule, and in the context of formulation, a dispersant or a stabilizer may be further employed.

Particularly, the pharmaceutical composition comprising the anti-Ang2 antibody or its antigen-binding fragment can be formulated into immunoliposomes. Liposomes comprising an antibody can be prepared using methods that are well-known in the art. The immunoliposomes may be produced from a lipid composition comprising phosphatidylcholine, cholesterol, and PEGylated phosphatidylethanolamine by reverse-phase evaporation. For example, Fab' can be conjugated to liposomes by disulfide reformation.

### EXAMPLES

Hereafter, the present invention will be described in detail by examples.

The following examples are intended merely to illustrate the invention and are not construed to restrict the invention.

### Example 1

### Preparation of a Mouse anti-Ang2 Antibody, 10D6

### 1.1. Immunization of a Mouse

A human Ang2 protein (R&D systems; 623-AN-025/CF) was administered to 5-week-old BALB/c mice along with an adjuvant to induce an immune response and hybridomas that produce an individual anti-Ang2 antibody were prepared according to the methods of Schwaber, et al (Schwaber, J and Cohen, E. P., “Human x Mouse Somatic Cell Hybrid Clones Secreting Immunoglobulins of Both Parental Types,” Nature, 244 (1973), 444-447).

More specifically, to obtain immunized mice necessary for developing hybridoma cell lines, 100 μg (microgram) of human Ang2 protein (R&D Systems) mixed with the same amount of a complete Freund's adjuvant was administered via an intraperitoneal injection to each of five 4˜6-week-old BALB/c mice (Japan SLC, Inc.). After two weeks, the antigen (half the previously injected amount) mixed with an incomplete Freund's adjuvant using the same method as described above was administered to each mouse via an intraperitoneal injection. After one additional week, a final boosting was performed and three days later, blood was collected from the tail of each mouse to obtain serum, which was then diluted at 1/1000 with PBS and subjected to an ELISA to verify that the titer of an antibody recognizing Ang2 was increased. From the results, mice in which a sufficient amount of the antibody was obtained were selected, and a cell fusion process was performed on the selected mice.

Three days before the cell fusion experiment, a mixture of 50 μg of PBS and 100 μg of human Ang2 protein (R&D systems) was administered via an intraperitoneal injection to BALB/c mice (Japan SLC, Inc.), and after each immunized mouse was anesthetized, its spleen located on the left side of the body was extracted. The extracted spleen was ground with a mesh to isolate cells, which were mixed with a culture medium (DMEM, Hyclon) to prepare a spleen cell suspension. The suspension was centrifuged to collect a cell layer. The obtained 1×10⁸ spleen cells were mixed with 1×10⁷ myeloma cells (Sp2/0), and the mixture was centrifuged to precipitate the cells. The centrifuged precipitate was slowly dispersed, treated with 1 ml of 45% polyethylene glycol (PEG 1500) contained in a culture medium (DMEM), and maintained at 37° C. for one minute before adding 1 ml of a culture medium (DMEM). Subsequently, 10 ml of the culture medium (DMEM) was added for 1 minute to the resultant, which was incubated in a water bath at 37° C. for 5 minutes and then re-centrifuged after the total volume was adjusted to 50 ml. The resulting cell precipitate was re-suspended in an isolation medium (HAT medium) at a concentration of 1˜2×10⁵/ml, and the resultant suspension was distributed at 0.1 ml to the each well of a 96-well plate, which was then incubated in a carbon dioxide incubator at 37° C. to prepare the hybridoma cell groups.

### 1.2. Production and Purification of a Monoclonal Antibody

The above obtained individual antibody producing hybridomas were screened using a typical ELISA format and 95 anti-Ang2 monoclonal antibodies among the hybridomas differentiated from their mother hybridomas were selected based on their binding potential with Ang2.

More specifically, to select the hybridoma cells that specifically react only to Ang2 protein among the hybridoma cell groups prepared in Example 1.1 above, an ELISA assay method using a human Ang2 protein as an antigen was used for screening.

Human Ang2 protein was added at 100 ng per each well to a microtiter plate to be adhered to the surface of the plate, and unreacted antigens were removed by washing. 50 microliters of the hybridoma cell culture obtained in Example 1 above was added to each well to react for 1 hour and then, the wells were sufficiently washed with phosphate buffered saline-TWEEN 20 (PBST) solution to remove unreacted culture solution. Goat anti-mouse IgG-horseradish peroxidase (goat anti-mouse IgG-HRP) was added thereto, a reaction was allowed to occur at a room temperature for 1 hour and then, washing was performed with the TBST solution. Subsequently, a substrate solution (OPD) of peroxidase was added to each well to react, and the reaction degree was measured by the absorption at 450 nm using an ELISA reader to repeatedly select hybridoma cell lines that secret antibodies having specifically high binding affinity only to human Ang2 protein. A limiting dilution was performed on the hybridoma cell lines obtained through repetitive selection to obtain final 58 clones of hybridoma cell lines producing monoclonal antibodies.

Each hybridoma obtained above was cultured in DMEM (Dulbeco's Modified Eagle's Medium) and then, the culture solutions were collected and subjected to Protein G-affinity chromatography to purify anti-Ang2 monoclonal antibodies produced from each hybridoma.

First, the hybridoma cells cultured in 50 ml of culture medium (DMEM) containing 10% (v/v) FBS were centrifuged to obtain a cell precipitate, which was washed at least twice with 20 ml of PBS to remove the FBS. The cell precipitate was re-suspended in 50 ml of the culture medium (DMEM) and then incubated in a carbon dioxide incubator at 37° C. for 3 days. Subsequently, the cell culture was centrifuged to remove the antibody-producing cells, and the culture medium including the secreted antibodies was isolated and then, stored at 4° C. or used directly. Antibodies were purified from 50 to 300 ml of the culture medium using an AKTA purification device (GE Healthcare) equipped with an affinity column (protein G agarose column; Pharmacia, USA). The purified antibodies were stored for subsequent use after replacing the supernatant with PBS using a filter for protein aggregation (Amicon), and used for the following examples.

### 1.3. Examination of Functions of Monoclonal Antibodies and Selection of Mouse anti-Ang2 Antibody 10D6

A test for analyzing an influence of the anti-Ang2 antibody on Tie2 phosphorylation was conducted using a cell-based assay.

HUVEC (ATCC) cells (1×10⁵ cells) were cultured in a 100 mm culture dish using EGM-2 (Lonza) media at 37° C. and when they reached 80˜90% confluency, the media were replaced with serum-free medium (Lonza) and cultured at 37° C. for 6 to 16 hours. The dish was washed once with PBS and after the replacement with 1 nM sodium orthovanadate (Sigma)-mixed serum free media (Lonza), they were further cultured for 10 min. After the cells were washed once again with PBS, the cultured cells were treated with a mixture prepared by mixing the anti-Ang2 antibody (10D6) having various concentrations (600˜0.06 nM) with 40 nM of Ang2 protein (R&D systems) and letting them stand for 20 min. and further cultured for 10 min.

The cells were washed using PBS, treated with 400 μl of a lysis buffer (Roche), collected to a tube to be dissolved at 4° C. for 30 min. and then, centrifuged at 13,000 rpm for 15 min. to measure a supernatant using Nanodrop. 1 μg of Tie2 antibody (R&D system) was added to 0.8 mg of a cell lysate, which was then overnight reacted at 4° C. and then subjected to immunoprecipitation by the addition of protein A bead (GE Healthcare) thereto. The obtained reactant was centrifuged at 13,000 rpm for 15 min. to obtain a pellet, which was washed two to three times with a lysis buffer (Roche), added to a sample buffer (Invitrogen) mixed with a reducing agent, and boiled at 95° C. for 5 min., and then, applied to NuPAGE Novex 4-12% Bis-Tris gel (Invitrogen) and transferred onto Nitrocellulose membrane (Invitrogen).

To examine the presence of the phosphorylation of Tie2, the membranes were blocked with PBST mixed with 3% (v/v) skim milk (Sigma) for 30 min. and identified using an HRP-conjugated anti-phospho tyrosine antibody (Millipore). For Tie2 identification, the blots were reacted in a stripping buffer (Thermo) for 15 min, then blocked again and identified using an anti-Tie2 antibody (Santa cruz). An antibody, which shows more intensive ability to induce a phosphorylation of Tie2 receptor when it is added together with Ang2 at the concentration of 60 nM, compared to the case treated with Ang2 only, was selected and named as 10D6.

The hybridoma producing 10D6 was deposited in the Korean Cell Line Bank located at Yongon-dong, Chongno-gu, Seoul, South Korea, as of Apr. 23, 2013 and received accession number KCLRF-BP-00295.

### 1.4. Analysis of Binding Affinity of Mouse Antibody 10D6 to Ang2

The binding affinity of the above antibody to human Ang2 protein was measured by an surface plasmon resonance (SPR) method using a BlAcore T100 (GE Healthcare). The SPR method uses refractive index change of light which passes a sensor chip according to the state of materials coated onto the sensor chip, and if an antigen or an antibody is flowed onto a chip coated with the antigen or antibody, it causes changes in refractive index due to their binding and Kd values are thus calculated from the measured values.

First, anti-His antibody was immobilized on a CMS sensor chip (GE healthcare) up to 8,000 RU levels using a pH 5.0 acetate solution and an amine coupling kit (GE Healthcare). 6 μg/m1 of a recombinant hAng2 (C-His, R&D

Systems) protein was flowed onto the chip to be captured at 100 to 200 RU levels. The antibody obtained in Example 2 above was diluted serially to twice each time starting from 100 nM concentration and it was each flowed onto the chip to allow it to be bound to (on), dissociated from (off), and regenerated (using 10 mM NaOH solution) from the antigen captured on the sensor chip, thereby to measure antigen-antibody affinity. With regard to hAng2, such experiments were conducted, and the results are as shown in the following Table 4.

TABLE 4

| antibody | hAng2 (Kd) |
|---|---|
| SAIT-ANG2-AB-m10D6 | 8.0 nM |

### Example 2

### Gene cloning of Mouse Antibody 10D6

RNA was obtained using RNeasy mini kit (Qiagen) from the antibody-producing hybridoma (2×10⁶ cells) obtained from Example 1.3 above. Then, by using the RNA as a template, only the gene sequence of the heavy chain and light chain variable regions of the monoclonal antibody to be produced in the hybridoma was amplified using a OneStep RT-PCR kit (Qiagen), a Mouse Ig-Primer Set (Novagen), and a thermocycler (GeneAmp PCR System 9700, Applied Biosystem) under the following conditions: 5 min. at 94° C.; [30 min. at 50° C., 15 min. at 95° C], [1 min. at 94° C., 1 min. at 50° C., 2 min. at 72° C.] x 35 cycles; 6 min. at 72° C.; cooling to 4° C.

The PCR products obtained from each reaction were subjected to a direct DNA sequencing to obtain the amino acid sequences of the CDR, heavy chain variable regions and light chain variable regions of the antibody, and nucleotide sequences encoding them, and the obtained results are set forth in the following Tables 5 to 8.

TABLE 5

| | Amino acid sequence of heavy | | |
|---|---|---|---|
| | chain CDR | | |
| | CDRH1- | CDRH2- | CDRH3- |
| Antibody | KABAT | KABAT | KABAT |
| SAIT-ANG2- | SDYAWN | YINYSGNTDYNPSLKS | GNFEGAMDY |
| AB-m10D6 | (SEQ ID | (SEQ ID NO: 2) | (SEQ ID |
| | NO: 1) | | NO: 3) |

TABLE 6

| | Amino acid sequence of light | | |
|---|---|---|---|
| | chain CDR | | |
| | CDRL1- | CDRL2- | CDRL3- |
| Antibody | KABAT | KABAT | KABAT |
| SAIT-ANG2- | KASQSVSNDVA | YASNRYP | QQDYSSPWT |
| AB-m10D6 | (SEQ ID | (SEQ ID | (SEQ ID |
| | NO: 4) | NO: 5) | NO: 6) |

TABLE 7

| Anti- | |
|---|---|
| body | Sequence of heavy chain variable region |
| SAIT- | DVQLQESGPDLVKPSQSLSLTCTVTGYSITSDYAWNWIRQFPGN |
| ANG2- | KLEWMGYINYSGNTDYNPSLKSRSSITRDTSKNQFFLQLNSVTT |
| AB- | GDTATYYCARGNFEGAMDYWGQGTSVTVSS(SEQ ID NO: 7) |
| m10D6 | |
| | GATGTGCAGCTTCAGGAGTCGGGACCTGACCTGGTGAAACCTTC |
| | TCAGTCTCTGTCCCTCACCTGCACTGTCACTGGCTACTCAATCAC |
| | CAGTGATTATGCCTGGAACTGGATCCGGCAGTTTCCAGGAAACA |
| | AACTGGAGTGGATGGGCTACATAAACTACAGTGGTAACACTGAC |
| | TACAACCCATCTCTCAAAAGTCGAAGCTCTATCACTCGAGACAC |
| | ATCCAAGAACCAGTTCTTCCTGCAGTTGAATTCTGTGACTACTGG |
| | GGACACAGCCACATATTACTGTGCAAGAGGTAACTTCGAAGGTG |
| | CTATGGACTACTGGGGTCAAGGAACCTCAGTCACCGTCTCCTCA |
| | (SEQ ID NO: 8) |

TABLE 8

| Antibody | Sequence of light chain variable region |
|---|---|
| SAIT- | SIVMTQTPKFLLVSAGDRVTITCKASQSVSNDVAWYQQK |
| ANG2- | PGQSPKLLIYYASNRYPGVPDRFTGSGYGTDFTFTISTV |
| AB- | QAEDLAVYFCQQDYSSPWTFGGGTKLEIK |
| m10D6 | (SEQ ID NO: 9) |
| | agtattgtgatgacccagactcccaaattcctgcttgta |
| | tcagcaggagacagggttaccataacctgcaaggccagt |
| | cagagtgtgagtaatgatgtagcttggtaccaacagaag |
| | ccagggcagtctcctaaactgctgatatactatgcatcc |
| | aatcgctaccctggagtccctgatcgcttcactggcagt |
| | ggatatgggacggatttcactttcaccatcagcactgtg |
| | caggctgaagacctggcagtttatttctgtcagcaggat |
| | tatagctctccgtggacgttcggtggaggcaccaagctg |
| | gaaatcaaa(SEQ ID NO: 10) |

(In above Tables 6 and 7, underlined bold letters are CDR1, CDR2, and CDR3 in sequence)

Based on the sequence information obtained above, single chain DNAs encoding the heavy chain variable region and the light chain variable region, respectively, were prepared, and cloned into vectors comprising a human kappa constant region coding gene and a CH1 region coding gene of human IgG1, respectively. In particular, a DNA fragment having the heavy chain variable region coding nucleotide sequence (SEQ ID NO: 8) was cloned into a vector of pOptiVEC™-TOPO TA Cloning Kit comprised in OptiCHO™ Antibody Express Kit (Cat no. 12762-019; Invitrogen), and a DNA fragment having the light chain variable region coding nucleotide sequence (SEQ ID NO: 10) was cloned into a vector of pcDNA™3.3-TOPO TA Cloning Kit(Cat no. 8300-01), using EcoRI(NEB, R0101S) and XhoI(NEB, R0146S), to construct a vector comprising the heavy chain variable region and a vector comprising the light chain variable region for expressing a chimeric antibody.

### Example 3:

### Preparation of an scFv of Mouse Antibody 10D6

A gene for producing an scFv fragment using the heavy chain variable region and the light chain variable region of mouse antibody 10D6 was designed. The heavy chain variable region (amino acid sequence: SEQ ID NO: 7; coding nucleotide sequence: SEQ ID NO: 8) and the light chain variable region (amino acid sequence: SEQ ID NO: 9; coding nucleotide sequence: SEQ ID NO: 10) were linked to form ‘VH-linker-VL’ construct, and the linker is designed to have the amino acid sequence of ‘GGGGSGGGGSGGGGS(SEQ ID NO: 76)’. The amino acid sequence of the designed ‘VH-linker-VL’ (scFv of 10D6) is represented in SEQ ID NO: 80 and the coding nucleotide sequence thereof is represented in SEQ ID NO: 81.

### Example 4

### Preparation of Gene Library for Affinity Maturation

### 4.1. Selection of target CDR and preparation of primers

To perform affinity maturation, six complementary determining regions (CDRs) were defined from the prepared mouse antibody 10D6 according to the ‘Kabat numbering’ rule. The CDRs are summarized in Table 9:

TABLE 9

| CDR | Amino acid sequence |
|---|---|
| CDR-H1 | SDYAWN(SEQ ID NO: 1) |
| CDR-H2 | YINYSGNTDYNPSLKS(SEQ ID NO: 2) |
| CDR-H3 | GNFEGAMDY(SEQ ID NO: 3) |
| CDR-L1 | KASQSVSNDVA(SEQ ID NO: 4) |
| CDR-L2 | YASNRYP(SEQ ID NO: 5) |
| CDR-L3 | QQDYSSPWT(SEQ ID NO: 6) |

For use in the introduction of random sequences into the CDRs of the antibody, primers were designed as follows. Conventionally, N codons were utilized to introduce bases at the same ratio (25% A, 25% G, 25% C, 25% T) into desired sites of mutation. In this experiment, the introduction of random bases into the CDRs of 10D6 was conducted in such a manner that, of the three nucleotides per codon in the wild-type polynucleotide encoding each CDR, the first and second nucleotides conserved over 85% of the entire sequence while the other three nucleotides were introduced at the same percentage (each 5%) and that the same possibility was imparted to the third nucleotide (33% G, 33% C, 33% T).

### 4.2. Construction of Gene Library of scFv of 10D6 Antibody

The construction of antibody gene libraries through the introduction of random sequences was carried out using the primers synthesized in the same manner as in Example 4.1. Two PCR products were obtained using a polynucleotide covering the 10D6 scFv (SEQ ID NO: 81) as a template (see following figure), and were subjected to overlap extension PCR to give scFv library genes for 10D6 antibodies in which only desired CDRs were mutated.

10⁷˜10⁸ libraries targeting each of the six CDRs prepared from the scFv library genes were constructed.

The affinity for Ang2 of each library was compared to that of the wild-type. Most libraries were lower in affinity for Ang2, compared to the wild-type. However, in some mutants, the affinity for Ang2 was retained.

### Example 5

### Selection of Antibody with Improved Affinity from Libraries

Among the scFv libraries provided in Example 4, the scFv fragments showing upper 1.0 percent of affinity to Ang-2 were selected, and this process was repeated four times. The nucleotide sequence of each of the selected scFv was analyzed. The obtained nucleotide sequences are summarized in Table 10, and were converted into IgG forms (a heavy chain constant region: constant region of human IgG1, a light chain constant region: constant region of human KAPPA Chain). Five antibodies which were respectively produced from clones VH-6.6, VH-6.7, VL-(6.11), VL-(6.17), and VL-HU1(6.22) were used in the subsequent experiments.

TABLE 10

| | Library | |
|---|---|---|
| clones | constructed | CDR sequence |
| VH-6.6 | CDR-H2 | KISYSGKTDYNPSLKS |
| | | (SEQ ID NO: 14) |
| VH-6.7 | CDR-H2 | KINYAGNTDYNPSLKS |
| | | (SEQ ID NO: 15) |
| VL-(6.11) | CDR-L1 | KASQSVSNDVH |
| | | (SEQ ID NO: 16) |
| VL-(6.17) | CDR-L3 | QHDYSSPFT |
| | | (SEQ ID NO: 19) |
| VL-(6.22) | CDR-L1 + | KASQSVSNDVH |
| | CDR-L3 | (SEQ ID NO: 16) + |
| | | QHDYSSPFT |
| | | (SEQ ID NO: 19) |

### Example 6

### Preparation of Humanized Antibody 10D6-HU1, 10D6-HU2, 10D6-HU3, and 10D6-HU5, from mouse antibody 10D6

### 6.1. Heavy Chain Humanization

To design three domains 10D6-HU1 Heavy, 10D6-HU2-heavy, and 10D6-HU5-heavy, human germline genes which share the highest identity/homology with the VH gene of the mouse antibody 10D6 purified were analyzed through an Ig BLAST (IgBLAST online database tool, maintained by National Center for Biotechnology Information (NCBI), Bethesda, Md.). The analysis results revealed that IGHV4-b*01 (DP-67; accession number: Z12367) has an identity/identity/homology of 72% at the amino acid level. CDR-H1(SEQ ID NO: 1), CDR-H2(SEQ ID NO: 2), and CDR-H3(SEQ ID NO: 3) of the mouse antibody 10D6 were defined according to Kabat numbering.

A design was made to introduce the CDR of the mouse antibody 10D6 into the framework of IGHV4-b*01 (named as 10D6-HU1; SEQ ID NO: 77; QVQLQESGPGLVKPSETLSLTCAVSGYSISSDYAWNWIRQPPGKGLEWIGYIN YSGNTDYNPSLKSRVTISVDTSKNQFSLKLSSVTAADTAVYYCARGNFEGAM DYWGQGTLVTVSS). Hereupon, a back mutation to the amino acid sequence of the mouse 10D6 were conducted at positions 30 (S→T), to establish antibody 10D6-HU5 (SEQ ID NO: 56). Then, 10D6-HU5 was further mutated at positions 48 (I→M), 67 (V→S), and 71 (V→R), to establish 10D6-HU2(SEQ ID NO: 78; QVQLQESGPGLVKP SETLSLTCAVSGYSITSDYAWNWIRQPPGKGLEWMGYI NYSGNTDYNPSLKSRSTISRDTSKNQFSLKLSSVTAADTAVYYCARGNFEGA MDYWGQGTLVTVSS).

For use in designing 10D6-HU3-heavy, human antibody frameworks were analyzed by a BLAST search. The result revealed that the Herceptin backbone, which known to show very low immunogenicity of about 0.1% level among the pre-existing humanized antibodies, is very similar in framework and sequence to the mouse antibody 10D6. CDR-H1, CDR-H2, and CDR-H3 of the mouse antibody 10D6 were defined according to Kabat numbering and introduced into the Herceptin backbone to construct H4-heavy (SEQ ID NO: 42), wherein back mutations were conducted at positions 27 (F-Y), 28(N→S), 30(K→T), 48(V→M), 49(A→G), 67 (F→S), 71(A→R), 78(A→F), and 93(S→A), to establish 10D6-HU3(SEQ ID NO: 79; EVQLVESGGGLVQPGGSLRLSCAASGYSITSDYAWNWVRQAPGKGLEWMG YINYSGNTDYNP SLKSRSTISRDTSKNTFYLQMNSLRAEDTAVYYCARGNFEG AMDYWGQGTLVTVSS).

### 6.2. Light Chain Humanization

To design a H1-light, human germline genes which share the highest identity/homology with the VL gene of the mouse antibody 10D6 were analyzed through an Ig BLAST. The analysis results revealed IGKV1-39*01(012; accession number: X59315) has an identity/identity/homology of 66% at the amino acid level. CDR-L1(SEQ ID NO: 4), CDR-L2(SEQ ID NO: 5), and CDR-L3(SEQ ID NO: 6) of the mouse antibody 10D6 were defined according to Kabat numbering. A design was made to introduce the CDR of the mouse antibody 10D6 into the framework of IGKV1-39*01.

Thereafter, DNA fragments of heavy chains (10D6-VHHU1, 10D6-VHHU2, 10D6-VHHU3, and 10D6-VHHU5) were respectively cloned into a vector of pOptiVEC™-TOPO TA Cloning Kit enclosed in an OptiCHO™ Antibody Express Kit (Cat no. 12762-019, Invitrogen) using EcoRI(NEB, R0101S) and nhel(NEB, R0131), and a DNA fragment of a light chain (10D6-VLHU1(SEQ ID NO: 57), coding sequence: SEQ ID NO: 69) was cloned into a vector of pcDNATM3.3-TOPO TA Cloning Kit using EcoRI(NEB, R0101S) and XhoI(NEB, R0146S), to construct recombinant vectors for expressing a humanized antibody.

The constructed vectors were amplified using a Qiagen Maxiprep kit (Cat No. 12662), and the vectors including the heavy chain and the vector including the light chain were added to 293T cells (2.5×10⁷) at a ratio of about 4:1 (about 80 ug:20 ug) with 360 ul of 2 M CaCl₂ and were transfected. Next, the mixture was cultured in a DMEM medium with 10% (w/v) FBS at 37° C. in 5% (v/v) CO₂ conditions for 5 hours, and then cultured in a DMEM medium without FBS at 37° C. in 5% (v/v) CO₂ conditions for 48 hours.

The cultured cells were centrifuged, and 100 ml of each supernatant was purified using AKTA Prime (GE healthcare). Protein A column (GE healthcare, 17-0405-03) was placed in the AKTA Prime, and the cultured solution was flowed at a flow rate of 5 ml/min and was eluted with IgG elution buffer (Thermo Scientific, 21004). The buffer was replaced with a PBS buffer, and thus final humanized antibodies 10D6-HU1, 10D6-HU2, 10D6-HU3, and 10D6-HU5 were purified.

### Example 7

### Incorporation of the Selected CDRs into Humanized Antibody and Transformation to IgG

The selected CDRs were incorporated into the heavy chain and the light chain of the humanized antibodies. Polynucleotides encoding the heavy chain of the antibodies were synthesized by Bioneer, Inc. so as to consist of ‘EcoRI-signal sequence-VH-NheI-CH-XhoI’ (SEQ ID NOs: 64-68). Polynucleotides encoding the light chain of the antibodies were synthesized by Bioneer, Inc. so as to consist of ‘EcoRI-signal sequence-VL-BsiWI-CL-XhoI’ (SEQ ID NOs: 69-71). The polynucleotides (SEQ ID NOs: 64-68) encoding the heavy chain were respectively cloned into a vector of pOptiVEC™-TOPO TA Cloning Kit included in OptiCHO™ Antibody Express Kit (Cat no. 12762-019; Invitrogen), and the polynucleotides (SEQ ID NOs: 69-71) encoding the light chain were respectively cloned into a vector of pcDNA™3.3-TOPOTA Cloning Kit(Cat no. 8300-01), using EcoRI(NEB, R0101S) and XhoI(NEB, R0146S), to establish vectors for expressing affinity matured antibodies.

The constructed vectors were amplified using a Qiagen Maxiprep kit (Cat No. 12662), and the vectors including the heavy chain and the vector including the light chain were added to 293T cells (2.5×10⁷) at a ratio of about 4:1 (about 80 ug:20 ug) with 360 ul of 2 M CaCl₂ and were transfected. Next, the mixture was cultured in a DMEM medium with 10% (w/v) FBS at 37° C. in 5% (v/v) CO₂ conditions for 5 hours, and then cultured in a DMEM medium without FBS at 37° C. in 5% (v/v) CO₂ conditions for 48 hours.

The cultured cells were centrifuged, and 100 ml of each supernatant was purified using AKTA Prime (GE healthcare). Protein A column (GE healthcare, 17-0405-03) was placed in the AKTA Prime, and the cultured solution was flowed at a flow rate of 5 ml/min and was eluted with IgG elution buffer (Thermo Scientific, 21004). The buffer was replaced with a PBS buffer, and thus final affinity-matured antibodies h10D6-Opti-1, h10D6-Opti-2, h10D6-Opti-3, and h10D6-Opti-4 were purified.

TABLE 11

| Clone | Antibody sequence (VH) | Antibody sequence (VL) |
|---|---|---|
| h10D6-OPTI-1 | >HU2-6.6 | >HU1 |
| | QVQLQESGPGLVKPSETLSLTCAVSGYS | DIQMTQSPSSLSASVGDRVTITCKASQSV |
| | ITSDYAWNWIRQPPGKGLEWMGKISYS | SNDVAWYQQKPGKAPKLLIYYASNRYP |
| | GKTDYNPSLKSRSTISRDTSKNQFSLKL | GVPSRFSGSGSGTDFTLTISSLQPEDFATY |
| | SSVTAADTAVYYCARGNFEGAMDYW | YCQQDYSSPWTFGQGTKLEIK |
| | GQGTLVTVSS(SEQ ID NO: 52) | (SEQ ID NO: 57) |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGCT | TCCCTGTCTGCATCTGTAGGAGACAGA |
| | CTCACTTACCTGTGCCGTTAGTGGATA | GTCACCATCACTTGCAAGGCCAGTCAG |
| | CTCTATCACTTCCGACTACGCTTGGAA | AGTGTGAGTAATGATGTAGCTTGGTAT |
| | TTGGATTCGGCAGCCTCCAGGCAAAG | CAGCAGAAACCAGGGAAAGCCCCTAA |
| | GGCTGGAATGGATGGGAAAGATTTCC | GCTCCTGATCTATTATGCATCCAATCGC |
| | TATTCCGGTAAGACTGACTACAATCCC | TACCCTGGGGTCCCATCAAGGTTCAGT |
| | AGTCTGAAGAGCAGGTCAACAATCTC | GGCAGTGGATCTGGGACAGATTTCACT |
| | CAGAGACACCAGCAAGAATCAGTTTT | CTCACCATCAGCAGTCTGCAACCTGAA |
| | CCCTGAAATTGTCCTCGGTGACAGCAG | GATTTTGCAACTTACTACTGTCAGCAG |
| | CGGATACCGCAGTGTATTATTGCGCCC | GATTATAGCTCTCCGTGGACGTTCGGT |
| | GCGGTAACTTCGAGGGAGCTATGGAT | GGAGGCACCAAGGTGGAAATCAAA |
| | TACTGGGGGCAGGGTACTCTCGTCACT | (SEQ ID NO: 69) |
| | GTGAGCAGC(SEQ ID NO: 64) | |
| h10D6-OPTI-2 | >HU2-6.7 | >HU1 |
| | QVQLQESGPGLVKPSETLSLTCAVSGYS | DIQMTQSPSSLSASVGDRVTITCKASQSV |
| | ITSDYAWNWIRQPPGKGLEWMGKINY | SNDVAWYQQKPGKAPKLLIYYASNRYP |
| | AGNTDYNPSLKSRSTISRDTSKNQFSLK | GVPSRFSGSGSGTDFTLTISSLQPEDFATY |
| | LSSVTAADTAVYYCARGNFEGAMDYW | YCQQDYSSPWTFGQGTKLEIK(SEQ ID |
| | GQGTLVTVSS(SEQ ID NO: 53) | NO: 57) |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGCT | TCCCTGTCTGCATCTGTAGGAGACAGA |
| | CTCACTTACCTGTGCCGTTAGTGGATA | GTCACCATCACTTGCAAGGCCAGTCAG |
| | CTCTATCACTTCCGACTACGCTTGGAA | AGTGTGAGTAATGATGTAGCTTGGTAT |
| | TTGGATTCGGCAGCCTCCAGGCAAAG | CAGCAGAAACCAGGGAAAGCCCCTAA |
| | GGCTGGAATGG | GCTCCTGATCTATTATGCATCCAATCGC |
| | ATGGGAAAGATTAACTATGCCGGTAA | TACCCTGGGGTCCCATCAAGGTTCAGT |
| | CACTGACTACAATCCCAGTCTGAAGA | GGCAGTGGATCTGGGACAGATTTCACT |
| | GCAGGTCAACAATCTCCAGAGACACC | CTCACCATCAGCAGTCTGCAACCTGAA |
| | AGCAAGAATCAGTTTTCCCTGAAATTG | GATTTTGCAACTTACTACTGTCAGCAG |
| | TCCTCGGTGACAGCAGCGGATACCGC | GATTATAGCTCTCCGTGGACGTTCGGT |
| | AGTGTATTATTGCGCCCGCGGTAACTT | GGAGGCACCAAGGTGGAAATCAAA |
| | CGAGGGAGCTATGGATTACTGG | (SEQ ID NO: 69) |
| | GGGCAGGGTACTCTCGTCACTGTGAG | |
| | CAGC(SEQ ID NO: 65) | |
| h10D6-OPTI-43 | >HU2-6.6 | >2HU1-6.11 |
| | QVQLQESGPGLVKPSETLSLTCAVSGYS | DIQMTQSPSSLSASVGDRVTITCKASQSV |
| | ITSDYAWNWIRQPPGKGLEWMGKISYS | SNDVHWYQQKPGKAPKLLIYYASNRYP |
| | GKTDYNPSLKSRSTISRDTSKNQFSLKL | GVPSRFSGSGSGTDFTLTISSLQPEDFATY |
| | SSVTAADTAVYYCARGNFEGAMDYW | YCQQDYSSPWTFGQGTKLEIK(SEQ ID |
| | GQGTLVTVSS(SEQ ID NO: 52) | NO: 58) |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGCT | TCCCTGTCTGCATCTGTAGGAGACAGA |
| | CTCACTTACCTGTGCCGTTAGTGGATA | GTCACCATCACTTGCAAGGCCAGTCAG |
| | CTCTATCACTTCCGACTACGCTTGGAA | AGTGTGAGTAATGATGTACATTGGTAT |
| | TTGGATTCGGCAGCCTCCAGGCAAAG | CAGCAGAAACCAGGGAAAGCCCCTAA |
| | GGCTGGAATGG | GCTCCTGATCTATTATGCATCCAATCGC |
| | ATGGGAAAGATTTCCTATTCCGGTAAG | TACCCTGGGGTCCCATCAAGGTTCAGT |
| | ACTGACTACAATCCCAGTCTGAAGAG | GGCAGTGGATCTGGGACAGATTTCACT |
| | CAGGTCAACAATCTCCAGAGACACCA | CTCACCATCAGCAGTCTGCAACCTGAA |
| | GCAAGAATCAGTTTTCCCTGAAATTGT | GATTTTGCAACTTACTACTGTCAGCAG |
| | CCTCGGTGACAGCAGCGGATACCGCA | GATTATAGCTCTCCGTGGACGTTCGGT |
| | GTGTATTATTGCGCCCGCGGTAACTTC | GGAGGCACCAAGGTGGAAATCAAA |
| | GAGGGAGCTATGGATTACTGG | (SEQ ID NO: 70) |
| | GGGCAGGGTACTCTCGTCACTGTGAG | |
| | CAGC(SEQ ID NO: 64) | |
| h10D6-OPTI-55 | >HU2-6.7 | HU1-6.11 |
| | QVQLQESGPGLVKPSETLSLTCAVSGYS | DIQMTQSPSSLSASVGDRVTITCKASQSV |
| | ITSDYAWNWIRQPPGKGLEWMGKINY | SNDVHWYQQKPGKAPKLLIYYASNRYP |
| | AGNTDYNPSLKSRSTISRDTSKNQFSLK | GVPSRFSGSGSGTDFTLTISSLQPEDFATY |
| | LSSVTAADTAVYYCARGNFEGAMDYW | YCQQDYSSPWTFGQGTKLEIK(SEQ ID |
| | GQGTLVTVSS(SEQ ID NO: 53) | NO: 58) |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGCT | TCCCTGTCTGCATCTGTAGGAGACAGA |
| | CTCACTTACCTGTGCCGTTAGTGGATA | GTCACCATCACTTGCAAGGCCAGTCAG |
| | CTCTATCACTTCCGACTACGCTTGGAA | AGTGTGAGTAATGATGTACATTGGTAT |
| | TTGGATTCGGCAGCCTCCAGGCAAAG | CAGCAGAAACCAGGGAAAGCCCCTAA |
| | GGCTGGAATGG | GCTCCTGATCTATTATGCATCCAATCGC |
| | ATGGGAAAGATTAACTATGCCGGTAA | TACCCTGGGGTCCCATCAAGGTTCAGT |
| | CACTGACTACAATCCCAGTCTGAAGA | GGCAGTGGATCTGGGACAGATTTCACT |
| | GCAGGTCAACAATCTCCAGAGACACC | CTCACCATCAGCAGTCTGCAACCTGAA |
| | AGCAAGAATCAGTTTTCCCTGAAATTG | GATTTTGCAACTTACTACTGTCAGCAG |
| | TCCTCGGTGACAGCAGCGGATACCGC | GATTATAGCTCTCCGTGGACGTTCGGT |
| | AGTGTATTATTGCGCCCGCGGTAACTT | GGAGGCACCAAGGTGGAAATCAAA |
| | CGAGGGAGCTATGGATTACTGG | (SEQ ID NO: 70) |
| | GGGCAGGGTACTCTCGTCACTGTGAG | |
| | CAGC(SEQ ID NO: 65) | |
| h10D6-OPTI-3 | >2HU3-6.6 | >HU1 |
| | EVQLVESGGGLVQPGGSLRLSCAASGY | DIQMTQSPSSLSASVGDRVTITCKASQSV |
| | SITSDYAWNWVRQAPGKGLEWMGKIS | SNDVAWYQQKPGKAPKLLIYYASNRYP |
| | YSGKTDYNPSLKSRSTISRDTSKNTFYL | GVPSRFSGSGSGTDFTLTISSLQPEDFATY |
| | QMNSLRAEDTAVYYCARGNFEGAMD | YCQQDYSSPWTFGQGTKLEIK(SEQ ID |
| | YWGQGTLVTVSS(SEQ ID NO: | NO: 57) |
| | 54) | |
| | (coding nucleotide sequence) | (coding nucleotide sequence) |
| | GAGGTTCAGCTGGTCGAAAGCGGTGG | GACATCCAGATGACCCAGTCTCCATCC |
| | GGGACTCGTGCAGCCAGGCGGTTCTCT | TCCCTGTCTGCATCTGTAGGAGACAGA |
| | TAGATTATCATGTGCCGCATCCGGGTA | GTCACCATCACTTGCAAGGCCAGTCAG |
| | CTCCATCACCTCTGATTATGCATGGAA | AGTGTGAGTAATGATGTAGCTTGGTAT |
| | CTGGGTCAGACAAGCCCCCGGAAAGG | CAGCAGAAACCAGGGAAAGCCCCTAA |
| | GCCTGGAGTGGATGGGGAAGATCTCC | GCTCCTGATCTATTATGCATCCAATCGC |
| | TATTCAGGGAAGACAGATTATAATCCT | TACCCTGGGGTCCCATCAAGGTTCAGT |
| | TCGCTGAAAAGCAGATCAACAATTAG | GGCAGTGGATCTGGGACAGATTTCACT |
| | TAGAGACACTTCTAAAAATACTTTTTA | CTCACCATCAGCAGTCTGCAACCTGAA |
| | CCTCCAGATGAACAGTCTGCGCGCCG | GATTTTGCAACTTACTACTGTCAGCAG |
| | AAGACACCGCCGTGTACTACTGCGCT | GATTATAGCTCTCCGTGGACGTTCGGT |
| | AGGGGAAATTTCGAGGGAGCTATGGA | GGAGGCACCAAGGTGGAAATCAAA |
| | CTATTGGGGCCAGGGCACGTTGGTAA | (SEQ ID NO: 69) |
| | CCGTGAGCAGC(SEQ ID NO: 66) | |
| h10D6-OPTI-4 | >HU3-6.7 | >HU1 |
| | EVQLVESGGGLVQPGGSLRLSCAASGY | DIQMTQSPSSLSASVGDRVTITCKASQSV |
| | SITSDYAWNWVRQAPGKGLEWMGKIN | SNDVAWYQQKPGKAPKLLIYYASNRYP |
| | YAGNTDYNPSLKSRSTISRDTSKNTFYL | GVPSRFSGSGSGTDFTLTISSLQPEDFATY |
| | QMNSLRAEDTAVYYCARGNFEGAMD | YCQQDYSSPWTFGQGTKLEIK(SEQ ID |
| | YWGQGTLVTVSS(SEQ ID NO: 55) | NO: 57) |
| | (coding nucleotide sequence) | (coding nucleotide sequence) |
| | GAGGTTCAACTGGTAGAGTCCGGGGG | GACATCCAGATGACCCAGTCTCCATCC |
| | CGGCCTGGTCCAGCCAGGAGGAAGCC | TCCCTGTCTGCATCTGTAGGAGACAGA |
| | TGCGGCTCTCTTGTGCCGCCAGCGGGT | GTCACCATCACTTGCAAGGCCAGTCAG |
| | ATAGTATCACTTCAGATTATGCCTGGA | AGTGTGAGTAATGATGTAGCTTGGTAT |
| | ATTGGGTCCGCCAGGCCCCCGGGAAG | CAGCAGAAACCAGGGAAAGCCCCTAA |
| | GGCTTAGAGTGGATGGGTAAAATTAA | GCTCCTGATCTATTATGCATCCAATCGC |
| | TTACGCAGGCAACACCGACTATAATC | TACCCTGGGGTCCCATCAAGGTTCAGT |
| | CTTCACTGAAATCTAGATCCACCATCT | GGCAGTGGATCTGGGACAGATTTCACT |
| | CTAGAGATACAAGTAAGAACACCTTT | CTCACCATCAGCAGTCTGCAACCTGAA |
| | TACTTGCAGATGAATAGCCTCAGGGCT | GATTTTGCAACTTACTACTGTCAGCAG |
| | GAAGACACTGCTGTGTACTACTGCGC | GATTATAGCTCTCCGTGGACGTTCGGT |
| | AAGAGGAAACTTCGAAGGAGCGATGG | GGAGGCACCAAGGTGGAAATCAAA |
| | ATTATTGGGGCCAGGGTACGCTTGTGA | (SEQ ID NO: 69) |
| | CAGTGTCCTCT(SEQ ID NO: 67) | |
| h10D6-OPTI-16 | >HU3-6.6 | >HU1-6.11 |
| | EVQLVESGGGLVQPGGSLRLSCAASGY | DIQMTQSPSSLSASVGDRVTITCKASQSV |
| | SITSDYAWNWVRQAPGKGLEWMGKIS | SNDVHWYQQKPGKAPKLLIYYASNRYP |
| | YSGKTDYNPSLKSRSTISRDTSKNTFYL | GVPSRFSGSGSGTDFTLTISSLQPEDFATY |
| | QMNSLRAEDTAVYYCARGNFEGAMD | YCQQDYSSPWTFGQGTKLEIK(SEQ ID |
| | YWGQGTLVTVSS(SEQ ID NO: 54) | NO: 58) |
| | (coding nucleotide sequence) | (coding nucleotide sequence) |
| | GAGGTTCAGCTGGTCGAAAGCGGTGG | GACATCCAGATGACCCAGTCTCCATCC |
| | GGGACTCGTGCAGCCAGGCGGTTCTCT | TCCCTGTCTGCATCTGTAGGAGACAGA |
| | TAGATTATCATGTGCCGCATCCGGGTA | GTCACCATCACTTGCAAGGCCAGTCAG |
| | CTCCATCACCTCTGATTATGCATGGAA | AGTGTGAGTAATGATGTACATTGGTAT |
| | CTGGGTCAGACAAGCCCCCGGAAAGG | CAGCAGAAACCAGGGAAAGCCCCTAA |
| | GCCTGGAGTGGATGGGGAAGATCTCC | GCTCCTGATCTATTATGCATCCAATCGC |
| | TATTCAGGGAAGACAGATTATAATCCT | TACCCTGGGGTCCCATCAAGGTTCAGT |
| | TCGCTGAAAAGCAGATCAACAATTAG | GGCAGTGGATCTGGGACAGATTTCACT |
| | TAGAGACACTTCTAAAAATACTTTTTA | CTCACCATCAGCAGTCTGCAACCTGAA |
| | CCTCCAGATGAACAGTCTGCGCGCCG | GATTTTGCAACTTACTACTGTCAGCAG |
| | AAGACACCGCCGTGTACTACTGCGCT | GATTATAGCTCTCCGTGGACGTTCGGT |
| | AGGGGAAATTTCGAGGGAGCTATGGA | GGAGGCACCAAGGTGGAAATCAAA |
| | CTATTGGGGCCAGGGCACGTTGGTAA | (SEQ ID NO: 70) |
| | CCGTGAGCAGC(SEQ | ID NO: 66) |
| h10D6-OPTI-17 | >HU3-6.7 | >HU1-6.11 |
| | EVQLVESGGGLVQPGGSLRLSCAASGY | DIQMTQSPSSLSASVGDRVTITCKASQSV |
| | SITSDYAWNWVRQAPGKGLEWMGKIN | SNDVHWYQQKPGKAPKLLIYYASNRYP |
| | YAGNTDYNPSLKSRSTISRDTSKNTFYL | GVPSRFSGSGSGTDFTLTISSLQPEDFATY |
| | QMNSLRAEDTAVYYCARGNFEGAMD | YCQQDYSSPWTFGQGTKLEIK(SEQ ID |
| | YWGQGTLVTVSS(SEQ ID NO: 55) | NO: 58) |
| | (coding nucleotide sequence) | (coding nucleotide sequence) |
| | GAGGTTCAACTGGTAGAGTCCGGGGG | GACATCCAGATGACCCAGTCTCCATCC |
| | CGGCCTGGTCCAGCCAGGAGGAAGCC | TCCCTGTCTGCATCTGTAGGAGACAGA |
| | TGCGGCTCTCTTGTGCCGCCAGCGGGT | GTCACCATCACTTGCAAGGCCAGTCAG |
| | ATAGTATCACTTCAGATTATGCCTGGA | AGTGTGAGTAATGATGTACATTGGTAT |
| | ATTGGGTCCGCCAGGCCCCCGGGAAG | CAGCAGAAACCAGGGAAAGCCCCTAA |
| | GGCTTAGAGTGGATGGGTAAAATTAA | GCTCCTGATCTATTATGCATCCAATCGC |
| | TTACGCAGGCAACACCGACTATAATC | TACCCTGGGGTCCCATCAAGGTTCAGT |
| | CTTCACTGAAATCTAGATCCACCATCT | GGCAGTGGATCTGGGACAGATTTCACT |
| | CTAGAGATACAAGTAAGAACACCTTT | CTCACCATCAGCAGTCTGCAACCTGAA |
| | TACTTGCAGATGAATAGCCTCAGGGCT | GATTTTGCAACTTACTACTGTCAGCAG |
| | GAAGACACTGCTGTGTACTACTGCGC | GATTATAGCTCTCCGTGGACGTTCGGT |
| | AAGAGGAAACTTCGAAGGAGCGATGG | GGAGGCACCAAGGTGGAAATCAAA |
| | ATTATTGGGGCCAGGGTACGCTTGTGA | (SEQ ID NO: 70) |
| | CAGTGTCCTCT(SEQ ID NO: 67) | |
| h10D6-OPTI-42 | >HU5 | >HU1-22 |
| | QVQLQESGPGLVKPSETLSLTCAVSGYS | DIQMTQSPSSLSASVGDRVTITCKASQSV |
| | ITSDYAWNWIRQPPGKGLEWIGYINYS | SNDVHWYQQKPGKAPKLLIYYASNRYP |
| | GNTDYNPSLKSRVTISVDTSKNQFSLKL | GVPSRFSGSGSGTDFTLTISSLQPEDFATY |
| | SSVTAADTAVYYCARGNFEGAMDYW | YCQHDYSSPFTFGQGTKLEIK(SEQ ID |
| | GQGTLVTVSS(SEQ ID NO: 56) | NO: 59) |
| | (coding nucleotide sequence) | (coding nucleotide sequence) |
| | CAGGTGCAGCTGCAGGAGTCGGGCCC | GACATCCAGATGACCCAGTCTCCATCC |
| | AGGACTGGTGAAGCCTTCGGAGACCC | TCCCTGTCTGCATCTGTAGGAGACAGA |
| | TGTCCCTCACCTGCGCTGTCTCTGGTT | GTCACCATCACTTGCAAGGCCAGTCAG |
| | ACTCCATCACCAGTGATTATGCCTGGA | AGTGTGAGTAATGATGTACATTGGTAT |
| | ACTGGATCCGGCAGCCCCCAGGGAAG | CAGCAGAAACCAGGGAAAGCCCCTAA |
| | GGGCTGGAGTGGATTGGGTACATAAA | GCTCCTGATCTATTATGCATCCAATCGC |
| | CTACAGTGGTAACACTGACTACAACC | TACCCTGGGGTCCCATCAAGGTTCAGT |
| | CATCTCTCAAAAGTCGAGTCACCATAT | GGCAGTGGATCTGGGACAGATTTCACT |
| | CAGTAGACACGTCCAAGAACCAGTTC | CTCACCATCAGCAGTCTGCAACCTGAA |
| | TCCCTGAAGCTGAGCTCTGTGACCGCC | GATTTTGCAACTTACTACTGTCAGCAT |
| | GCAGACACGGCCGTGTATTACTGTGC | GATTATAGCTCTCCGTTCACGTTCGGTG |
| | GAGAGGTAACTTCGAAGGTGCTATGG | GAGGCACCAAGGTGGAAATCAAA(SEQ |
| | ACTACTGGGGTCAAGGAACGCTTGTG | ID NO: 71) |
| | ACAGTGTCCTCT(SEQ ID NO: 68) | |

(In Table 11, the bold letters are CDR1, CDR2, and CDR3 in sequence)

### Example 8

### Analysis of Binding Affinity of Selected Antibodies

The binding affinity (KD values) of the antibodies to human Ang2 protein was measured by an SPR method using a BlAcore T100 (GE Healthcare). 25 μg/m1 anti-His antibody was immobilized on a CM5 sensor chip (GE healthcare) using a pH 5.0 acetate solution and an amine coupling kit (GE Healthcare). 6 μg/ml of a recombinant hAng2 (C-His, R&D Systems) protein was flowed onto the chip to be captured at 100 to 200 RU levels. The antibodies obtained in the above examples were diluted serially to twice each time starting from 100 nM concentration and it was each flowed onto the chip to allow it to be bound to (on), dissociated from (off), and regenerated (using 10 mM NaOH solution) from the antigen captured on the sensor chip, thereby to measure antigen-antibody affinity. The KD values were calculated from the values of kₒₙ, k_{off}, and the results are as shown in the following Table 12.

TABLE 12

| Antibody | kon (1/Ms) | koff (1/s) | KD (nM) |
|---|---|---|---|
| m10D6 | 2.410 × 10⁴ | 1.932 × 10⁻⁴ | 8 |
| 10D6-HU1 | 3.082 × 10⁴ | 0.002599 | 84 |
| 10D6-HU2 | 7.298 × 10⁴ | 0.003464 | 47 |
| 10D6-HU3 | 4.503 × 10⁴ | 0.001938 | 43 |
| 10D6-HU5 | 4.856 × 10⁴ | 0.003115 | 64 |
| h10D6-OPTI-1 | 4.737 × 10⁵ | 3.209 × 10⁻⁴ | 0.68 |
| h10D6-OPTI-2 | 4.237 × 10⁵ | 1.488 × 10⁻⁴ | 0.34 |
| h10D6-OPTI-43 | 1.531 × 10⁶ | 5.760 × 10⁻⁴ | 0.38 |
| h10D6-OPTI-55 | 6.210 × 10⁵ | 8.489 × 10⁻⁵ | 0.14 |
| h10D6-OPTI-3 | 6.239 × 10⁵ | 3.070 × 10⁻⁴ | 0.49 |
| h10D6-OPTI-4 | 7.357 × 10⁵ | 2.460 × 10⁻⁴ | 0.33 |
| h10D6-OPTI-16 | 4.794 × 10⁵ | 4.434 × 10⁻⁴ | 0.92 |
| h10D6-OPTI-17 | 4.600 × 10⁵ | 3.503 × 10⁻⁴ | 0.76 |
| h10D6-OPTI-42 | 3.358 × 10⁵ | 2.862 × 10⁻⁴ | 0.85 |

As shown in Table 12, the affinity to Ang2 of the mouse antibody 10D6 is about 8 nM, the affinities to Ang2 of the 5 affinity-matured and humanized antibodies are from about 0.14 nM to about 0.92 nM. The results indicate that the affinity to Ang2 can be improved at least about 5 times up to about 37 times in the affinity-matured antibodies in an IgG form transformed from a scFv form.

### Example 9

### Synthesis of a Polynucleotide for Preparing an scFv of Humanized Antibody of 10D6 (Opti-1)

The gene for preparing scFv of a humanized 10D6 antibody was designed using the heavy chain variable region and the light chain variable region of humanized 10D6 antibody Opti-1. The heavy chain variable region (amino acid sequence: Hu2 6.6(SEQ ID NO: 52); coding nucleotide sequence: SEQ ID NO: 64), and the light chain variable region(amino acid sequence: SEQ ID NO: Hu1 (SEQ ID NO: 57); coding nucleotide sequence: SEQ ID NO: SEQ ID NO: 69) were linked to form a ‘VH-linker-VL’ construct, and the linker was designed so as to have the amino acid sequence of ‘GGGGSGGGGSGGGGS(SEQ ID NO: 76)’. The polynucleotide (SEQ ID NO: 83) encoding the designed scFv ('VH-linker-VL'; SEQ ID NO: 82) of antibody 10D6 opti-1 was synthesized by Bioneer, Inc.

### Example 10

### Preparation of Gene Library for the Secondary Affinity Maturation

### 10.1. Selection of Target CDR and Preparation of Primers

To perform affinity maturation of antibody 10D6 opti-1, three complementary determining regions (CDRs) were defined from the prepared antibody 10D6 opti-1 according to the ‘Kabat numbering’ rule. The CDRs are summarized in Table 13:

TABLE 13

| CDR | amino acid sequence |
|---|---|
| CDR-L1 | KASQSVSNDVA(SEQ ID NO: 4) |
| CDR-L2 | YASNRYP(SEQ ID NO: 5) |
| CDR-L3 | QQDYSSPWT(SEQ ID NO: 6) |

For use in the introduction of random sequences into the CDRs of the antibody, primers were designed as follows. Conventionally, N codons were utilized to introduce bases at the same ratio (25% A, 25% G, 25% C, 25% T) into desired sites of mutation. In this experiment, the introduction of random bases into the CDRs of 10D6 was conducted in such a manner that, of the three nucleotides per codon in the wild-type polynucleotide encoding each CDR, the first and second nucleotides conserved over 85% of the entire sequence while the other three nucleotides were introduced at the same percentage (each 5%) and that the same possibility was imparted to the third nucleotide (33% G, 33% C, 33% T).

### 10.2. Construction of Gene Library of scFv of 10D6 opti-1 Antibody

The construction of antibody gene libraries through the introduction of random sequences was carried out using the primers synthesized in the same manner as in Example 11.1. Two PCR products were obtained using a polynucleotide covering the 10D6 opti-1 scFv(SEQ ID NO: 83) as a template (see following figure), and were subjected to overlap extension PCR to give scFv library genes for 10D6 antibodies in which only desired CDRs were mutated.

10⁷˜10⁸ libraries targeting each of the six CDRs prepared from the scFv library genes were constructed.

The affinity for Ang2 of each library was compared to that of the wild-type. Most libraries were lower in affinity for Ang2, compared to the wild-type. However, in some mutants, the affinity for Ang2 was retained.

### Example 11

### Selection of Antibody with Improved Affinity from Libraries

Among the scFv libraries provided in Example 11, the scFv fragments showing upper 1.0 percent of affinity to Ang-2 were selected, and this process was repeated four times. The nucleotide sequence of each of the selected scFv was analyzed. The obtained nucleotide sequences are summarized in Table 14, and were converted into IgG forms (a heavy chain constant region: constant region of human IgG1, a light chain constant region: constant region of human KAPPA Chain). Four antibodies which were respectively produced from clones 10D6_VL-Hu1-2.1, 10D6_VL-Hu1-2.4, 10D6_VL-Hu1-2.7, 10D6_VL-Hu1-2.8 were used in the subsequent experiments.

TABLE 14

| | Library | |
|---|---|---|
| Clones | constructed | CDR sequence |
| 10D6_VL-Hu1-2.1 | CDR-L1 | KASQFVSTDVH |
| | | (SEQ ID NO: 17) |
| 10D6_VL-Hu1-2.4 | CDR-L2 | YASIPYP |
| | | (SEQ ID NO: 18) |
| 10D6_VL-Hu1-2.7 | CDR-L1 + L2 | KASQSVSNDVH |
| | | (SEQ ID NO: 16) + |
| | | YASIPYP |
| | | (SEQ ID NO: 18) |
| 10D6_VL-Hu1-2.8 | CDR-L1 + L2 | KASQFVSTDVH |
| | | (SEQ ID NO: XX) + |
| | | YASIPYP |
| | | (SEQ ID NO: 18) |

### Example 12

### Incorporation of the Selected CDRs into Humanized Antibody and Transformation to IgG

The selected CDRs were incorporated into the heavy chain and the light chain of the humanized antibodies. The heavy chain was derived from the antibody cloned with Hu2-6.6 or Hu3-6.6. Polynucleotides encoding the light chain of the antibodies were synthesized by Bioneer, Inc. so as to consist of ‘EcoRI-signal sequence-VL-BsiWI-CL-XhoI’ (see Table 15). The polynucleotides encoding the heavy chain were respectively cloned into a vector of pOptiVEC™-TOPO TA Cloning Kit included in OptiCHO™ Antibody Express Kit (Cat no. 12762-019; Invitrogen), and the polynucleotides encoding the light chain were respectively cloned into a vector of pcDNA™3.3-TOPOTA Cloning Kit(Cat no. 8300-01), using EcoRI(NEB, R0101S) and XhoI(NEB, R0146S), to establish vectors for expressing affinity matured antibodies.

The constructed vectors were amplified using a Qiagen Maxiprep kit (Cat No. 12662), and the vectors including the heavy chain and the vector including the light chain were added to 293T cells (2.5×10⁷) at a ratio of about 4:1 (about 80 ug:20 ug) with 360 ul of 2 M CaCl₂ and were transfected. Next, the mixture was cultured in a DMEM medium with 10% (w/v) FBS at 37° C. in 5% (v/v) CO₂ conditions for 5 hours, and then cultured in a DMEM medium without FBS at 37° C. in 5% (v/v) CO₂ conditions for 48 hours.

The cultured cells were centrifuged, and 100 ml of each supernatant was purified using AKTA Prime (GE healthcare). Protein A column (GE healthcare, 17-0405-03) was placed in the AKTA Prime, and the cultured solution was flowed at a flow rate of 5 ml/min and was eluted with IgG elution buffer (Thermo Scientific, 21004). The buffer was replaced with a PBS buffer, and thus final affinity-matured antibodies (hereinafter, named as h10D6-Opti-63, h10D6-Opti-64, h10D6-Opti-65, h10D6-Opti-66, h10D6-Opti-67, h10D6-Opti-71, h10D6-Opti-68, h10D6-Opti-70, h10D6-Opti-72, and h10D6-Opti-73) were purified.

TABLE 15

| Clone | Antibody sequence (VH) | Antibody sequence (VL) |
|---|---|---|
| h10D6-OPTI-63 | >HU2-6.6 | >10D6_VL-Hu1-2.1 |
| | QVQLQESGPGLVKPSETLSLTCAVSGYS | DIQMTQSPSSLSASVGDRVTITCKASQFV |
| | ITSDYAWNWIRQPPGKGLEWMGKISYS | STDVHWYQQKPGKAPKLLIYYASNRYP |
| | GKTDYNPSLKSRSTISRDTSKNQFSLKL | GVPSRFSGSGSGTDFTLTISSLQPEDFATY |
| | SSVTAADTAVYYCARGNFEGAMDYW | YCQQDYSSPWTFGQGTKLEIK(SEQ ID |
| | GQGTLVTVSS(SEQ ID NO: 52) | NO: 60) |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGC | TCCCTGTCTGCATCTGTAGGAGACAGA |
| | TCTCACTTACCTGTGCCGTTAGTGGAT | GTCACCATCACTTGCAAGGCCAGTCAG |
| | ACTCTATCACTTCCGACTACGCTTGGA | TTCGTGAGTACTGATGTACATTGGTAT |
| | ATTGGATTCGGCAGCCTCCAGGCAAA | CAGCAGAAACCAGGGAAAGCCCCTAA |
| | GGGCTGGAATGG | GCTCCTGATCTATTATGCATCCAATCGC |
| | ATGGGAAAGATTTCCTATTCCGGTAA | TACCCTGGGGTCCCATCAAGGTTCAGT |
| | GACTGACTACAATCCCAGTCTGAAGA | GGCAGTGGATCTGGGACAGATTTCACT |
| | GCAGGTCAACAATCTCCAGAGACACC | CTCACCATCAGCAGTCTGCAACCTGAA |
| | AGCAAGAATCAGTTTTCCCTGAAATT | GATTTTGCAACTTACTACTGTCAGCAG |
| | GTCCTCGGTGACAGCAGCGGATACCG | GATTATAGCTCTCCGTGGACGTTCGGT |
| | CAGTGTATTATTGCGCCCGCGGTAAC | GGAGGCACCAAGGTGGAAATCAAA |
| | TTCGAGGGAGCTATGGATTACTGG | (SEQ ID NO: 72) |
| | GGGCAGGGTACTCTCGTCACTGTGAG | |
| | CAGC(SEQ ID NO: 64) | |
| h10D6-OPTI-64 | >HU2-6.6 | DIQMTQSPSSLSASVGDRVTITCKASQSV |
| | QVQLQESGPGLVKPSETLSLTCAVSGYS | SNDVAWYQQKPGKAPKLLIYYASNRYP |
| | ITSDYAWNWIRQPPGKGLEWMGKISYS | GVPSRFSGSGSGTDFTLTISSLQPEDFATY |
| | GKTDYNPSLKSRSTISRDTSKNQFSLKL | YCGQDYASPWTFGQGTKLEIK(SEQ ID |
| | SSVTAADTAVYYCARGNFEGAMDYW | NO: 87) |
| | GQGTLVTVSS(SEQ ID NO: 52) | |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGC | TCCCTGTCTGCATCTGTAGGAGACAGA |
| | TCTCACTTACCTGTGCCGTTAGTGGAT | GTCACCATCACTTGCAAGGCCAGTCAG |
| | ACTCTATCACTTCCGACTACGCTTGGA | AGTGTGAGTAATGATGTAGCT |
| | ATTGGATTCGGCAGCCTCCAGGCAAA | TGGTATCAGCAGAAACCAGGGAAAGC |
| | GGGCTGGAATGG | CCCTAAGCTCCTGATCTATTATGCATCC |
| | ATGGGAAAGATTTCCTATTCCGGTAA | AACCGATACCCTGGGGTCCCATCAAGG |
| | GACTGACTACAATCCCAGTCTGAAGA | TTCAGTGGCAGTGGATCTGGGACAGAT |
| | GCAGGTCAACAATCTCCAGAGACACC | TTCACTCTCACCATCAGCAGTCTGCAA |
| | AGCAAGAATCAGTTTTCCCTGAAATT | CCTGAAGATTTTGCAACTTACTACTGT |
| | GTCCTCGGTGACAGCAGCGGATACCG | GGACAGGATTATGCCTCTCCGTGGACG |
| | CAGTGTATTATTGCGCCCGCGGTAAC | TTCGGTGGAGGCACCAAGGTGGAAATC |
| | TTCGAGGGAGCTATGGATTACTGG | AAA(SEQ ID NO: 88) |
| | GGGCAGGGTACTCTCGTCACTGTGAG | |
| | CAGC(SEQ ID NO: 64) | |
| h10D6-OPTI-65 | >HU2-6.6 | >10D6_VL-Hu1-2.4 |
| | QVQLQESGPGLVKPSETLSLTCAVSGYS | DIQMTQSPSSLSASVGDRVTITCKASQSV |
| | ITSDYAWNWIRQPPGKGLEWMGKISYS | SNDVAWYQQKPGKAPKLLIYYASIPYPG |
| | GKTDYNPSLKSRSTISRDTSKNQFSLKL | VPSRFSGSGSGTDFTLTISSLQPEDFATYY |
| | SSVTAADTAVYYCARGNFEGAMDYW | CQQDYSSPWTFGQGTKLEIK(SEQ ID |
| | GQGTLVTVSS(SEQ ID NO: 52) | NO: 61) |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGC | TCCCTGTCTGCATCTGTAGGAGACAGA |
| | TCTCACTTACCTGTGCCGTTAGTGGAT | GTCACCATCACTTGCAAGGCCAGTCAG |
| | ACTCTATCACTTCCGACTACGCTTGGA | AGTGTGAGTAATGATGTAGCTTGGTAT |
| | ATTGGATTCGGCAGCCTCCAGGCAAA | CAGCAGAAACCAGGGAAAGCCCCTAA |
| | GGGCTGGAATGG | GCTCCTGATCTATTATGCATCCATCCCA |
| | ATGGGAAAGATTTCCTATTCCGGTAA | TACCCTGGGGTCCCATCAAGGTTCAGT |
| | GACTGACTACAATCCCAGTCTGAAGA | GGCAGTGGATCTGGGACAGATTTCACT |
| | GCAGGTCAACAATCTCCAGAGACACC | CTCACCATCAGCAGTCTGCAACCTGAA |
| | AGCAAGAATCAGTTTTCCCTGAAATT | GATTTTGCAACTTACTACTGTCAGCAG |
| | GTCCTCGGTGACAGCAGCGGATACCG | GATTATAGCTCTCCGTGGACGTTCGGT |
| | CAGTGTATTATTGCGCCCGCGGTAAC | GGAGGCACCAAGGTGGAAATCAAA |
| | TTCGAGGGAGCTATGGATTACTGG | (SEQ ID NO: 73) |
| | GGGCAGGGTACTCTCGTCACTGTGAG | |
| | CAGC(SEQ ID NO: 64) | |
| h10D6-OPTI-66 | >HU2-6.6 | DIQMTQSPSSLSASVGDRVTITCKASQSV |
| | QVQLQESGPGLVKPSETLSLTCAVSGYS | SNDVHWYQQKPGKAPKLLIYYASNRYP |
| | ITSDYAWNWIRQPPGKGLEWMGKISYS | GVPSRFSGSGSGTDFTLTISSLQPEDFATY |
| | GKTDYNPSLKSRSTISRDTSKNQFSLKL | YCGQDYSAPWTFGQGTKLEIK(SEQ ID |
| | SSVTAADTAVYYCARGNFEGAMDYW | NO: 89) |
| | GQGTLVTVSS(SEQ ID NO: 52) | |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGC | TCCCTGTCTGCATCTGTAGGAGACAGA |
| | TCTCACTTACCTGTGCCGTTAGTGGAT | GTCACCATCACTTGCAAGGCCAGTCAG |
| | ACTCTATCACTTCCGACTACGCTTGGA | AGTGTGAGTAATGATGTACAC |
| | ATTGGATTCGGCAGCCTCCAGGCAAA | TGGTATCAGCAGAAACCAGGGAAAGC |
| | GGGCTGGAATGG | CCCTAAGCTCCTGATCTATTATGCATCC |
| | ATGGGAAAGATTTCCTATTCCGGTAA | AACCGATACCCTGGGGTCCCATCAAGG |
| | GACTGACTACAATCCCAGTCTGAAGA | TTCAGTGGCAGTGGATCTGGGACAGAT |
| | GCAGGTCAACAATCTCCAGAGACACC | TTCACTCTCACCATCAGCAGTCTGCAA |
| | AGCAAGAATCAGTTTTCCCTGAAATT | CCTGAAGATTTTGCAACTTACTACTGT |
| | GTCCTCGGTGACAGCAGCGGATACCG | GGACAGGATTATTCTGCCCCGTGGACG |
| | CAGTGTATTATTGCGCCCGCGGTAAC | TTCGGTGGAGGCACCAAGGTGGAAATC |
| | TTCGAGGGAGCTATGGATTACTGG | AAA(SEQ ID NO: 90) |
| | GGGCAGGGTACTCTCGTCACTGTGAG | |
| | CAGC(SEQ ID NO: 64) | |
| h10D6-OPTI-67 | >HU2-6.6 | >10D6_VL-Hu1-2.7 |
| | QVQLQESGPGLVKPSETLSLTCAVSGYS | DIQMTQSPSSLSASVGDRVTITCKASQSV |
| | ITSDYAWNWIRQPPGKGLEWMGKISYS | SNDVHWYQQKPGKAPKLLIYYASIPYPG |
| | GKTDYNPSLKSRSTISRDTSKNQFSLKL | VPSRFSGSGSGTDFTLTISSLQPEDFATYY |
| | SSVTAADTAVYYCARGNFEGAMDYW | CQQDYSSPWTFGQGTKLEIK(SEQ ID |
| | GQGTLVTVSS(SEQ ID NO: 52) | NO: 62) |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGC | TCCCTGTCTGCATCTGTAGGAGACAGA |
| | TCTCACTTACCTGTGCCGTTAGTGGAT | GTCACCATCACTTGCAAGGCCAGTCAG |
| | ACTCTATCACTTCCGACTACGCTTGGA | AGTGTGAGTAATGATGTACATTGGTAT |
| | ATTGGATTCGGCAGCCTCCAGGCAAA | CAGCAGAAACCAGGGAAAGCCCCTAA |
| | GGGCTGGAATGG | GCTCCTGATCTATTATGCATCCATCCCA |
| | ATGGGAAAGATTTCCTATTCCGGTAA | TACCCTGGGGTCCCATCAAGGTTCAGT |
| | GACTGACTACAATCCCAGTCTGAAGA | GGCAGTGGATCTGGGACAGATTTCACT |
| | GCAGGTCAACAATCTCCAGAGACACC | CTCACCATCAGCAGTCTGCAACCTGAA |
| | AGCAAGAATCAGTTTTCCCTGAAATT | GATTTTGCAACTTACTACTGTCAGCAG |
| | GTCCTCGGTGACAGCAGCGGATACCG | GATTATAGCTCTCCGTGGACGTTCGGT |
| | CAGTGTATTATTGCGCCCGCGGTAAC | GGAGGCACCAAGGTGGAAATCAAA |
| | TTCGAGGGAGCTATGGATTACTGG | (SEQ ID NO: 74) |
| | GGGCAGGGTACTCTCGTCACTGTGAG | |
| | CAGC(SEQ ID NO: 64) | |
| h10D6-OPTI-71 | >HU2-6.6 | 10D6_VL-Hu1-2.8 |
| | QVQLQESGPGLVKPSETLSLTCAVSGYS | DIQMTQSPSSLSASVGDRVTITCKASQFV |
| | ITSDYAWNWIRQPPGKGLEWMGKISYS | STDVHWYQQKPGKAPKLLIYYASIPYPG |
| | GKTDYNPSLKSRSTISRDTSKNQFSLKL | VPSRFSGSGSGTDFTLTISSLQPEDFATYY |
| | SSVTAADTAVYYCARGNFEGAMDYW | CQQDYSSPWTFGQGTKLEIK(SEQ ID |
| | GQGTLVTVSS(SEQ ID NO: 52) | NO: 63) |
| | (coding nucleotide sequence) | (coding nucleotide sequence) |
| | CAGGTGCAACTGCAGGAGTCAGGCCC | GACATCCAGATGACCCAGTCTCCATCC |
| | CGGCCTGGTAAAACCTTCTGAAACGC | TCCCTGTCTGCATCTGTAGGAGACAGA |
| | TCTCACTTACCTGTGCCGTTAGTGGAT | GTCACCATCACTTGCAAGGCCAGTCAG |
| | ACTCTATCACTTCCGACTACGCTTGGA | TTCGTGAGTACTGATGTACATTGGTAT |
| | ATTGGATTCGGCAGCCTCCAGGCAAA | CAGCAGAAACCAGGGAAAGCCCCTAA |
| | GGGCTGGAATGG | GCTCCTGATCTATTATGCATCCATCCCA |
| | ATGGGAAAGATTTCCTATTCCGGTAA | TACCCTGGGGTCCCATCAAGGTTCAGT |
| | GACTGACTACAATCCCAGTCTGAAGA | GGCAGTGGATCTGGGACAGATTTCACT |
| | GCAGGTCAACAATCTCCAGAGACACC | CTCACCATCAGCAGTCTGCAACCTGAA |
| | AGCAAGAATCAGTTTTCCCTGAAATT | GATTTTGCAACTTACTACTGTCAGCAG |
| | GTCCTCGGTGACAGCAGCGGATACCG | GATTATAGCTCTCCGTGGACGTTCGGT |
| | CAGTGTATTATTGCGCCCGCGGTAAC | GGAGGCACCAAGGTGGAAATCAAA |
| | TTCGAGGGAGCTATGGATTACTGG | (SEQ ID NO: 75) |
| | GGGCAGGGTACTCTCGTCACTGTGAG | |
| | CAGC(SEQ ID NO: 64) | |
| h10D6-OPTI-68 | >HU3-6.6 | >10D6_VL-Hu1-2.1 |
| | EVQLVESGGGLVQPGGSLRLSCAASGY | DIQMTQSPSSLSASVGDRVTITCKASQFV |
| | SITSDYAWNWVRQAPGKGLEWMGKIS | STDVHWYQQKPGKAPKLLIYYASNRYP |
| | YSGKTDYNPSLKSRSTISRDTSKNTFYL | GVPSRFSGSGSGTDFTLTISSLQPEDFATY |
| | QMNSLRAEDTAVYYCARGNFEGAMD | YCQQDYSSPWTFGQGTKLEIK(SEQ ID |
| | YWGQGTLVTVSS(SEQ ID NO: 54) | NO: 60) |
| | (coding nucleotide sequence) | (coding nucleotide sequence) |
| | GAGGTTCAGCTGGTCGAAAGCGGTGG | GACATCCAGATGACCCAGTCTCCATCC |
| | GGGACTCGTGCAGCCAGGCGGTTCTC | TCCCTGTCTGCATCTGTAGGAGACAGA |
| | TTAGATTATCATGTGCCGCATCCGGGT | GTCACCATCACTTGCAAGGCCAGTCAG |
| | ACTCCATCACCTCTGATTATGCATGGA | TTCGTGAGTACTGATGTACATTGGTAT |
| | ACTGGGTCAGACAAGCCCCCGGAAAG | CAGCAGAAACCAGGGAAAGCCCCTAA |
| | GGCCTGGAGTGGATGGGGAAGATCTC | GCTCCTGATCTATTATGCATCCAATCGC |
| | CTATTCAGGGAAGACAGATTATAATC | TACCCTGGGGTCCCATCAAGGTTCAGT |
| | CTTCGCTGAAAAGCAGATCAACAATT | GGCAGTGGATCTGGGACAGATTTCACT |
| | AGTAGAGACACTTCTAAAAATACTTT | CTCACCATCAGCAGTCTGCAACCTGAA |
| | TTACCTCCAGATGAACAGTCTGCGCG | GATTTTGCAACTTACTACTGTCAGCAG |
| | CCGAAGACACCGCCGTGTACTACTGC | GATTATAGCTCTCCGTGGACGTTCGGT |
| | GCTAGGGGAAATTTCGAGGGAGCTAT | GGAGGCACCAAGGTGGAAATCAAA |
| | GGACTATTGGGGCCAGGGCACGTTGG | (SEQ ID NO: 72) |
| | TAACCGTGAGCAGC(SEQ ID | |
| | NO: 66) | |
| h10D6-OPTI-70 | >HU3-6.6 | >10D6_VL-Hu1-2.4 |
| | EVQLVESGGGLVQPGGSLRLSCAASGY | DIQMTQSPSSLSASVGDRVTITCKASQSV |
| | SITSDYAWNWVRQAPGKGLEWMGKIS | SNDVAWYQQKPGKAPKLLIYYASIPYPG |
| | YSGKTDYNPSLKSRSTISRDTSKNTFYL | VPSRFSGSGSGTDFTLTISSLQPEDFATYY |
| | QMNSLRAEDTAVYYCARGNFEGAMD | CQQDYSSPWTFGQGTKLEIK(SEQ ID |
| | YWGQGTLVTVSS(SEQ ID NO: 54) | NO: 61) |
| | (coding nucleotide sequence) | (coding nucleotide sequence) |
| | GAGGTTCAGCTGGTCGAAAGCGGTGG | GACATCCAGATGACCCAGTCTCCATCC |
| | GGGACTCGTGCAGCCAGGCGGTTCTC | TCCCTGTCTGCATCTGTAGGAGACAGA |
| | TTAGATTATCATGTGCCGCATCCGGGT | GTCACCATCACTTGCAAGGCCAGTCAG |
| | ACTCCATCACCTCTGATTATGCATGGA | AGTGTGAGTAATGATGTAGCTTGGTAT |
| | ACTGGGTCAGACAAGCCCCCGGAAAG | CAGCAGAAACCAGGGAAAGCCCCTAA |
| | GGCCTGGAGTGGATGGGGAAGATCTC | GCTCCTGATCTATTATGCATCCATCCCA |
| | CTATTCAGGGAAGACAGATTATAATC | TACCCTGGGGTCCCATCAAGGTTCAGT |
| | CTTCGCTGAAAAGCAGATCAACAATT | GGCAGTGGATCTGGGACAGATTTCACT |
| | AGTAGAGACACTTCTAAAAATACTTT | CTCACCATCAGCAGTCTGCAACCTGAA |
| | TTACCTCCAGATGAACAGTCTGCGCG | GATTTTGCAACTTACTACTGTCAGCAG |
| | CCGAAGACACCGCCGTGTACTACTGC | GATTATAGCTCTCCGTGGACGTTCGGT |
| | GCTAGGGGAAATTTCGAGGGAGCTAT | GGAGGCACCAAGGTGGAAATCAAA |
| | GGACTATTGGGGCCAGGGCACGTTGG | (SEQ ID NO: 73) |
| | TAACCGTGAGCAGC(SEQ ID | |
| | NO: 66) | |
| h10D6-OPTI-72 | >HU3-6.6 | >10D6_VL-Hu1-2.7 |
| | EVQLVESGGGLVQPGGSLRLSCAASGY | DIQMTQSPSSLSASVGDRVTITCKASQSV |
| | SITSDYAWNWVRQAPGKGLEWMGKIS | SNDVHWYQQKPGKAPKLLIYYASIPYPG |
| | YSGKTDYNPSLKSRSTISRDTSKNTFYL | VPSRFSGSGSGTDFTLTISSLQPEDFATYY |
| | QMNSLRAEDTAVYYCARGNFEGAMD | CQQDYSSPWTFGQGTKLEIK(SEQ ID |
| | YWGQGTLVTVSS(SEQ ID NO: 54) | NO: 62) |
| | (coding nucleotide sequence) | (coding nucleotide sequence) |
| | GAGGTTCAGCTGGTCGAAAGCGGTGG | GACATCCAGATGACCCAGTCTCCATCC |
| | GGGACTCGTGCAGCCAGGCGGTTCTC | TCCCTGTCTGCATCTGTAGGAGACAGA |
| | TTAGATTATCATGTGCCGCATCCGGGT | GTCACCATCACTTGCAAGGCCAGTCAG |
| | ACTCCATCACCTCTGATTATGCATGGA | AGTGTGAGTAATGATGTACATTGGTAT |
| | ACTGGGTCAGACAAGCCCCCGGAAAG | CAGCAGAAACCAGGGAAAGCCCCTAA |
| | GGCCTGGAGTGGATGGGGAAGATCTC | GCTCCTGATCTATTATGCATCCATCCCA |
| | CTATTCAGGGAAGACAGATTATAATC | TACCCTGGGGTCCCATCAAGGTTCAGT |
| | CTTCGCTGAAAAGCAGATCAACAATT | GGCAGTGGATCTGGGACAGATTTCACT |
| | AGTAGAGACACTTCTAAAAATACTTT | CTCACCATCAGCAGTCTGCAACCTGAA |
| | TTACCTCCAGATGAACAGTCTGCGCG | GATTTTGCAACTTACTACTGTCAGCAG |
| | CCGAAGACACCGCCGTGTACTACTGC | GATTATAGCTCTCCGTGGACGTTCGGT |
| | GCTAGGGGAAATTTCGAGGGAGCTAT | GGAGGCACCAAGGTGGAAATCAAA |
| | GGACTATTGGGGCCAGGGCACGTTGG | (SEQ ID NO: 74) |
| | TAACCGTGAGCAGC(SEQ ID | |
| | NO: 66) | |
| h10D6-OPTI-73 | >HU3-6.6 | 10D6_VL-Hu1-2.8 |
| | EVQLVESGGGLVQPGGSLRLSCAASGY | DIQMTQSPSSLSASVGDRVTITCKASQFV |
| | SITSDYAWNWVRQAPGKGLEWMGKIS | STDVHWYQQKPGKAPKLLIYYASIPYPG |
| | YSGKTDYNPSLKSRSTISRDTSKNTFYL | VPSRFSGSGSGTDFTLTISSLQPEDFATYY |
| | QMNSLRAEDTAVYYCARGNFEGAMD | CQQDYSSPWTFGQGTKLEIK(SEQ ID |
| | YWGQGTLVTVSS(SEQ ID NO: 54) | NO: 63) |
| | (coding nucleotide sequence) | (coding nucleotide sequence) |
| | GAGGTTCAGCTGGTCGAAAGCGGTGG | GACATCCAGATGACCCAGTCTCCATCC |
| | GGGACTCGTGCAGCCAGGCGGTTCTC | TCCCTGTCTGCATCTGTAGGAGACAGA |
| | TTAGATTATCATGTGCCGCATCCGGGT | GTCACCATCACTTGCAAGGCCAGTCAG |
| | ACTCCATCACCTCTGATTATGCATGGA | TTCGTGAGTACTGATGTACATTGGTAT |
| | ACTGGGTCAGACAAGCCCCCGGAAAG | CAGCAGAAACCAGGGAAAGCCCCTAA |
| | GGCCTGGAGTGGATGGGGAAGATCTC | GCTCCTGATCTATTATGCATCCATCCCA |
| | CTATTCAGGGAAGACAGATTATAATC | TACCCTGGGGTCCCATCAAGGTTCAGT |
| | CTTCGCTGAAAAGCAGATCAACAATT | GGCAGTGGATCTGGGACAGATTTCACT |
| | AGTAGAGACACTTCTAAAAATACTTT | CTCACCATCAGCAGTCTGCAACCTGAA |
| | TTACCTCCAGATGAACAGTCTGCGCG | GATTTTGCAACTTACTACTGTCAGCAG |
| | CCGAAGACACCGCCGTGTACTACTGC | GATTATAGCTCTCCGTGGACGTTCGGT |
| | GCTAGGGGAAATTTCGAGGGAGCTAT | GGAGGCACCAAGGTGGAAATCAAA |
| | GGACTATTGGGGCCAGGGCACGTTGG | (SEQ ID NO: 75) |
| | TAACCGTGAGCAGC(SEQ ID | |
| | NO: 66) | |

(In Table 15, the bold letters are CDR1, CDR2, and CDR3 in sequence)

### Example 13

### Analysis of Binding Affinity of Selected Antibodies

The binding affinity (KD values) of the antibodies to human Ang2 protein was measured by an SPR method using a BIAcore T100 (GE Healthcare). 25 μg/ml anti-His antibody was immobilized on a CM5 sensor chip (GE healthcare) using a pH 5.0 acetate solution and an amine coupling kit (GE Healthcare). 6 μg/ml of a recombinant hAng2 (C-His, R&D Systems) protein was flowed onto the chip to be captured at 100 to 200 RU levels. The antibodies obtained in the above examples were diluted serially to twice each time starting from 100 nM concentration and it was each flowed onto the chip to allow it to be bound to (on), dissociated from (off), and regenerated (using 10 mM NaOH solution) from the antigen captured on the sensor chip, thereby to measure antigen-antibody affinity. The KD values were calculated from the values of kₒₙ k_{off}, and the results are as shown in the following Table 16.

TABLE 16

| Antibody | kon (1/Ms) | koff (1/s) | KD (M) |
|---|---|---|---|
| h10D6-OPTI-63 | 2.676 × 10⁶ | 7.421 × 10⁻⁵ | 2.773 × 10⁻¹¹ |
| h10D6-OPTI-65 | 4.960 × 16⁵ | 2.250 × 10⁻⁵ | 4.536 × 10⁻¹² |
| h10D6-OPTI-67 | 2.080 × 16⁶ | 2.684 × 10⁻⁷ | 1.291 × 10⁻¹³ |
| h10D6-OPTI-68 | 5.355 × 10⁵ | 1.696 × 10⁻⁴ | 3.168 × 10⁻¹⁰ |
| h10D6-OPTI-70 | 2.650 × 10⁵ | 1.159 × 10⁻⁴ | 4.374 × 10⁻¹⁰ |

As shown in Table 16, all the the affinity-matured and humanized antibodies show high affinity to Ang2 from about 0.000129 nM to about 0.43 nM.

### Example 14

### Preventive Effect of Anti-Ang2 Antibody against Thymus Involution as Verified by Thymus Size

Thymus involution is linked to senescence, and is found abundantly in patients with immune-related diseases such as acquired immune deficiency syndrome. To examine the effect of the anti-Ang2 antibody on thymus involution, the following experiments were preformed. Of the anti-Ang2 antibodies that were selected for affinity in Examples 1 to 13, the h10D6-Opti-67 antibody was illustratively used.

Mice at 4 months (young) and 24 months (old) of age were intraperitoneally injected once a week with an anti-Ang2 antibody (h10D6-Opti-67) or a control antibody (human IgG1) at a dose of 4 mg/kg for 8 weeks. Young and old mice injected with the control antibody were expressed as YC and OC, respectively, while the old mice injected with the anti-Ang2 antibody were denoted as OA. Eight weeks after antibody injection, the thymuses were isolated from the controls (YC, OC) and the experimental group (OA) and measured for morphology and size.

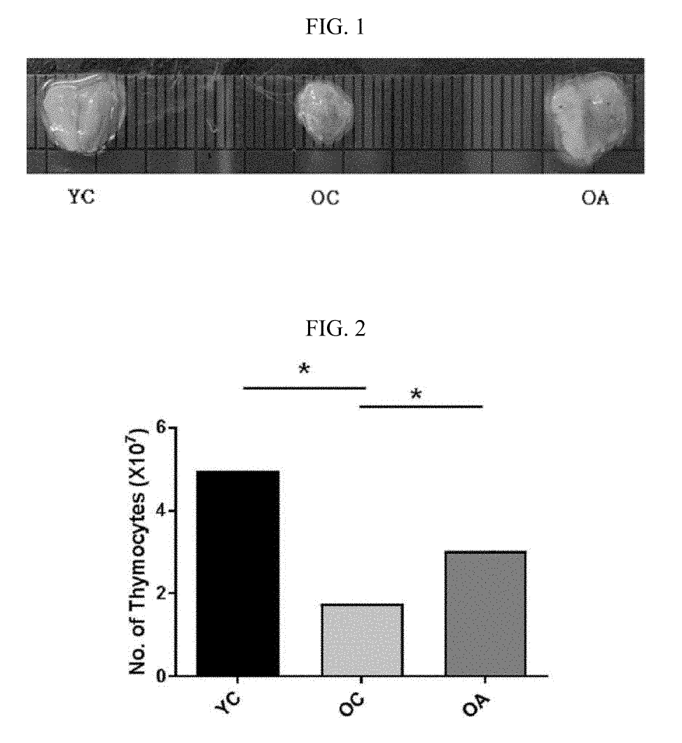

The results are depicted in FIG. 1. As can be seen in FIG. 1, the control antibody-treated old mice (OC) showed a reduction in thymus size of 50% or greater compared to the control antibody-treated young mice (YC), whereas the thymi from the old mice injected with the anti-Ang2 antibody (OA) were almost as large as those from the control young mice. These results indicate that the anti-Ang2 antibody is effective for preventing thymus involution.

### Example 15

### Preventive Effect of Anti-Ang2 Antibody against Thymus involution as Verified by Thymocyte Count

Thymus involution caused by senescence or immune-related diseases comprising AIDS causes a decrease in thymocyte count (number of hematopoietic stem cells in the thymus). In order to examine the effect of the anti-Ang2 antibody on thymocyte count, thymocytes were collected from the thymuses of the same mice as in Example 14 (YC, OC, OA; intraperitoneally injected once a week with the h10D6-Opti-67 antibody or the control antibody in a dose of 4 mg/kg for 8 weeks) and counted.

The results are depicted in FIG. 2 (mean values of experiments run in triplicate). As can be seen in FIG. 2, the control antibody-treated old mice (OC) were greatly reduced in thymocyte count compared to the control antibody-treated young mice (YC), whereas the old mice injected with the anti-Ang2 antibody (OA) showed a statistically significant increase in thymocyte count.

### Example 16

### Ameliorative Effect of Anti-Ang2 Antibody on Thymic Function

Spleens were isolated from the same mouse groups as in Example 14 (YC, OC, OA; intraperitoneally injected once a week with the h10D6-Opti-67 antibody or the control antibody at a dose of 4 mg/kg for 8 weeks), and analyzed for the level of naïve CD4+T cells, which play an important role in the implementation of thymic functions, by flow cytometry (LSRFortess flow cytometry, BD) using CD4, CD3, CD44, and CD62L markers (antibodies; all purchased from Abcam).

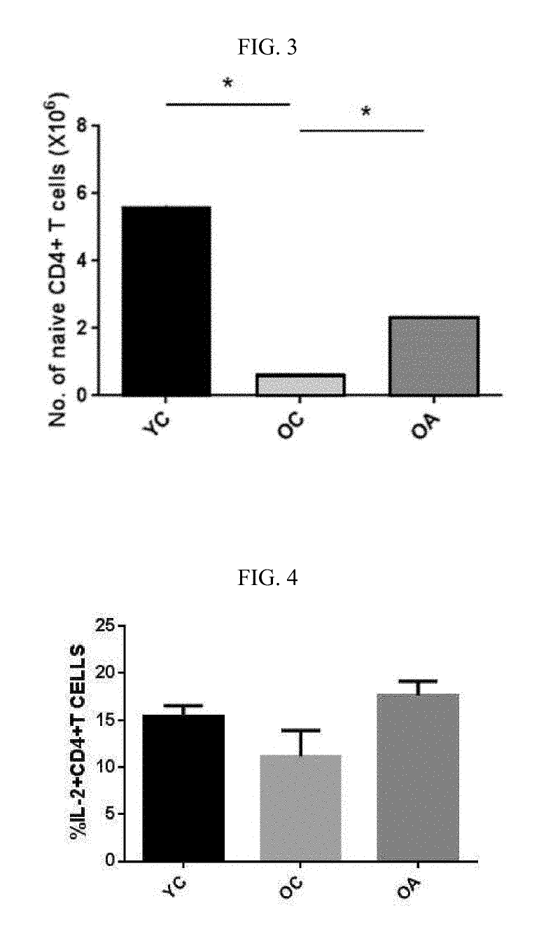

The results are depicted in FIG. 3 (mean values of experiments run in triplicate). As is understood from the data of FIG. 3, a sharp increase in the naive CD4+T cell count was detected in the control antibody-treated old mice (OC) compared to the control antibody-treated young mice (YC), whereas an increase was observed in the anti-Ang2 antibody-treated old mice (OA), demonstrating that the anti-Ang2 antibody ameliorates thymic function.

### Example 17

### Restorative Effect of Anti-Ang2 Antibody on Immune Function (Peripheral T Cell Function)

The IL-2 expression of CD4+T cells is down-regulated with senescence or in patients with immune-related diseases such as AIDS. IL-2 is representative of the cytokines that promote the proliferation of lymphocytes to thus enhance immune function.

Levels of CD4+T cells secreting the cytokine IL-2 in the same mouse groups as in Example 14 (YC, OC, OA; intraperitoneally injected once a week with the h10D6-Opti-67 antibody or the control antibody at a dose of 4 mg/kg for 8 weeks) were analyzed. Following immunostaining with anti-IL-2 antibody (Abcam), IL-2+, CD4+T cells were counted via flow cytometry (LSRFortess flow cytometry, BD).

Ratios of the IL-2-secreating CD4+T cells (%; No of IL-2+, CD4+T cells / No of total CD4+T cells) are depicted in FIG. 4. As shown in FIG. 4, the ability of CD4+T cells to produce IL-2 was degraded in the control antibody-treated old mice

(OC) compared to the control antibody-treated young mice (YC), but was restored in the anti-Ang2 antibody-treated old mice (OA).

### Example 18

### Promotive Effect of Anti-Ang2 Antibody on Self-Renewal of Hematopoietic Stem Cells

Myelocytes were isolated from the hind limb bones of the same mouse groups as in Example 14 (YC, OC, OA; intraperitoneally injected once a week with the h10D6-Opti-67 antibody or the control antibody in a dose of 4 mg/kg for 8 weeks) and were measured for % of hematopoietic stem cells therein by flow cytometry (LSRFortess flow cytometry, BD). Particularly, the percentage of self-renewal hematopoietic stem cells (long-term hematopoietic stem cells; LT-HSC), which is a main factor for T cell differentiation, was measured by immunostaining using lineage negative (CD3-, B220-, CD11b-, Ter119-, Gr1-), c-Kit, Flk2, and CD34 makers (Abeam).

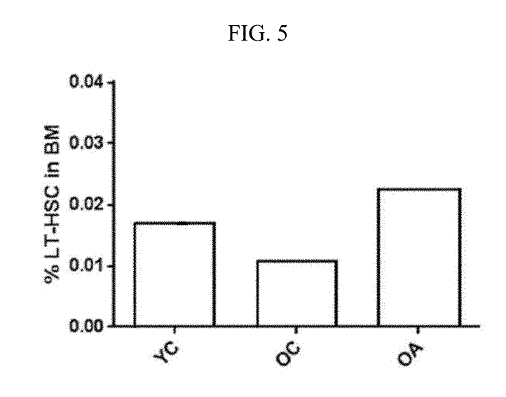

The measured ratios of self-renewal hematopoietic stem cells (%; No. of long-term hematopoietic stem cells/No. of total hematopoietic stem cells) are depicted in FIG. 5 (mean values of experiments run in triplicate). As shown in FIG. 5, the level of self-renewal hematopoietic stem cells was increased in the anti-Ang2 antibody-treated old mice (OA), compared to the control antibody-treated groups (YC, OC).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of potentiating immunity, comprising administering an anti-Ang2 antibody or an antigen-binding fragment thereof to a subject in need of immunopotentiation, wherein the anti-Ang2 antibody or antigen-binding fragment thereof binds to Ang2 and forms a complex with Tie2 receptor through Ang2.

2. The method of claim 1, wherein the anti-Ang2 antibody or antigen-binding fragment thereof binds to Q418, P419, a combination of Q418 and P419, or 2 to 20 consecutive acid residues of human Ang2 of SEQ ID NO: 11, including Q418, P419, a combination of Q418 or P419.

3. The method of claim 2, wherein the anti-Ang2 antibody or antigen-binding fragment thereof comprises:
a heavy chain complementarity-determining region H1 (CDR-H1) comprising SEQ ID NO: 1, a CDR-H2 comprising SEQ ID NO: 20, and a CDR-H3 comprising SEQ ID NO: 3,;
a light heavy chain complementarity-determining region-L1 (CDR-L1) comprising SEQ ID NO: 21, a CDR-L2 comprising SEQ ID NO: 22, and a CDR-L3 comprising SEQ ID NO: 23.

4. The method of claim 3, wherein the anti-Ang2 antibody or antigen-binding fragment thereof comprises:
a CDR-H1 comprising SEQ ID NO: 1, a CDR-H2 comprising SEQ ID NO: 2, 14 or 15, and a CDR-H3 comprising SEQ ID NO: 3;
a CDR-L1 comprising SEQ ID NO: 4, 16, or 17, a CDR-L2 comprising SEQ ID NO: 5 or 18, and a CDR-L3 comprising SEQ ID NO: 6 or 19.

5. The method of claim 4, wherein the anti-Ang2 antibody or antigen-binding fragment thereof comprises:
a heavy chain variable region comprising SEQ ID NO: 7, 52, 53, 54, 55, or 56; and
a light chain variable region comprising SEQ ID NO: 8, 57, 58, 59, 60, 61, 62, 63, 87, or 89.

6. The method of claim 1, wherein the anti-Ang2 antibody or antigen-binding fragment thereof is a mouse antibody, a chimeric antibody, or a humanized antibody.

7. The method of claim 1, wherein the immunopotentiation comprises:
improvement or increase in resistance to thymus involution, thymic function, the number of T cells, or production of hematopoietic stem cells, or augmentation or recovery of T cell functions.

8. A method of preventing or treating an immune-related disease, comprising administering an anti-Ang2 antibody or an antigen-binding fragment thereof to a subject in need of preventing or treating an immune-related disease, wherein the anti-Ang2 antibody or antigen-binding fragment thereof binds to Ang2 and forms a complex with Tie2 receptor through Ang2.

9. The method of claim 8, wherein the anti-Ang2 antibody or antigen-binding fragment thereof binds to Q418, P419, a combination of Q418 and P419, or 2 to 20 consecutive acid residues of human Ang2 of SEQ ID NO: 11, including Q418, P419, a combination of Q418 or P419.

10. The method of claim 9, wherein the anti-Ang2 antibody or antigen-binding fragment thereof comprises:
a CDR-H1 comprising SEQ ID NO: 1, a CDR-H2 comprising SEQ ID NO: 20, and a CDR-H3 comprising SEQ ID NO: 3;
a CDR-L1 comprising SEQ ID NO: 21, a CDR-L2 comprising SEQ ID NO: 22, and a CDR-L3 comprising SEQ ID NO: 23.

11. The method of claim 10, wherein the anti-Ang2 antibody or antigen-binding fragment thereof comprises:
a CDR-H1 comprising SEQ ID NO: 1, a CDR-H2 comprising SEQ ID NO: 2, 14 or 15, and a CDR-H3 comprising SEQ ID NO: 3;
a CDR-L1 comprising SEQ ID NO: 4, 16, or 17, a CDR-L2 comprising SEQ ID NO: 5 or 18, and a CDR-L3 comprising SEQ ID NO: 6 or 19.

12. The method of claim 11, wherein the anti-Ang2 antibody or antigen-binding fragment thereof comprises:
a heavy chain variable region comprising SEQ ID NO: 7, 52, 53, 54, 55, or 56; and
a light chain variable region comprising SEQ ID NO: 8, 57, 58, 59, 60, 61, 62, 63, 87, or 89.

13. The method of claim 8, wherein the anti-Ang2 antibody or antigen-binding fragment thereof is a mouse antibody, a chimeric antibody, or a humanized antibody.

14. The method of claim 8, wherein the immune-related disease is acquired immune deficiency syndrome or an autoimmune disease.


SEQUENCE LISTING (90 sequences)

SEQ ID NO: 1 - protein, 6 aa, Artificial Sequence
  Synthetic CDR-H1 of anti-Ang2 antibody
SDYAWN

SEQ ID NO: 2 - protein, 16 aa, Artificial Sequence
  Synthetic CDR-H2 of anti-Ang2 antibody
YINYSGNTDYNPSLKS

SEQ ID NO: 3 - protein, 9 aa, Artificial Sequence
  Synthetic CDR-H3 of anti-Ang2 antibody
GNFEGAMDY

SEQ ID NO: 4 - protein, 11 aa, Artificial Sequence
  Synthetic CDR-L1 of anti-Ang2 antibody
KASQSVSNDVA

SEQ ID NO: 5 - protein, 7 aa, Artificial Sequence
  Synthetic CDR-L2 of anti-Ang2 antibody
YASNRYP

SEQ ID NO: 6 - protein, 9 aa, Artificial Sequence
  Synthetic CDR-L3 of anti-Ang2 antibody
QQDYSSPWT

SEQ ID NO: 7 - protein, 118 aa, Artificial Sequence
  Synthetic Amino acid sequence of heavy chain variable region
DVQLQESGPDLVKPSQSLSLTCTVTGYSITSDYAWNWIRQFPGNKLEWMGYINYSGNTDYNPSLKSRSSITRDTSKNQFFLQLNSVTTGDTATYYCARGNFEGAMDYWGQGTSVTVSS

SEQ ID NO: 8 - DNA, 354 bp, Artificial Sequence
  Synthetic Nucleotide sequence encoding heavy chain variable region
gatgtgcagcttcaggagtcgggacctgacctggtgaaaccttctcagtctctgtccctcacctgcactgtcactggctactcaatcaccagtgattatgcctggaactggatccggcagtttccaggaaacaaactggagtggatgggctacataaactacagtggtaacactgactacaacccatctctcaaaagtcgaagctctatcactcgagacacatccaagaaccagttcttcctgcagttgaattctgtgactactggggacacagccacatattactgtgcaagaggtaacttcgaaggtgctatggactactggggtcaaggaacctcagtcaccgtctcctca

SEQ ID NO: 9 - protein, 107 aa, Artificial Sequence
  Synthetic Amino acid sequence of light chain variable region
SIVMTQTPKFLLVSAGDRVTITCKASQSVSNDVAWYQQKPGQSPKLLIYYASNRYPGVPDRFTGSGYGTDFTFTISTVQAEDLAVYFCQQDYSSPWTFGGGTKLEIK

SEQ ID NO: 10 - DNA, 321 bp, Artificial Sequence
  Synthetic Nucleotide sequence emcoding light chain variable region
agtattgtgatgacccagactcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctaccctggagtccctgatcgcttcactggcagtggatatgggacggatttcactttcaccatcagcactgtgcaggctgaagacctggcagtttatttctgtcagcaggattatagctctccgtggacgttcggtggaggcaccaagctggaaatcaaa

SEQ ID NO: 11 - protein, 496 aa, Artificial Sequence
  Synthetic Human Ang2
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 12 - protein, 121 aa, Artificial Sequence
  Synthetic Amino acid sequence of heavy chain variable region (4H10)
EVQLLESGGGLVQPGGSLRLSCAASGFTFSSYDMSWVRQAPGKGLEWVSLISPDSSSIYYADSVKGRFTISRDNSKNTLYLQMNSLRAEDTAVYYCAKDLISFWRGGFDYWGQGTLVTVSS

SEQ ID NO: 13 - protein, 111 aa, Artificial Sequence
  Synthetic Amino acid sequence of light chain variable region (4H10)
QSVLTQPPSASGTPGQRVTISCSGSSSNIGSNYVNWYQQLPGTAPKLLIYADSNRPSGVPDRFSGSKSGTSASLAISGLRSEDEADYYCGSWDYSLSGYVFGGGTKLTVLG

SEQ ID NO: 14 - protein, 16 aa, Artificial Sequence
  Synthetic CDR-H2 of anti-Ang2 antibody
KISYSGKTDYNPSLKS

SEQ ID NO: 15 - protein, 16 aa, Artificial Sequence
  Synthetic CDR-H2 of anti-Ang2 antibody
KINYAGNTDYNPSLKS

SEQ ID NO: 16 - protein, 11 aa, Artificial Sequence
  Synthetic CDR-L1 of anti-Ang2 antibody
KASQSVSNDVH

SEQ ID NO: 17 - protein, 11 aa, Artificial Sequence
  Synthetic CDR-L1 of anti-Ang2 antibody
KASQFVSTDVH

SEQ ID NO: 18 - protein, 7 aa, Artificial Sequence
  Synthetic CDR-L2 of anti-Ang2 antibody
YASIPYP

SEQ ID NO: 19 - protein, 9 aa, Artificial Sequence
  Synthetic CDR-L3 of anti-Ang2 antibody
QHDYSSPFT

SEQ ID NO: 20 - protein, 16 aa, Artificial Sequence
  Synthetic CDR-H2 of anti-Ang2 antibody
XIXYXGXTDYNPSLKS

SEQ ID NO: 21 - protein, 11 aa, Artificial Sequence
  Synthetic CDR-L1 of anti-Ang2 antibody
KASQXVSXDVX

SEQ ID NO: 22 - protein, 7 aa, Artificial Sequence
  Synthetic CDR-L2 of anti-Ang2 antibody
YASXXYP

SEQ ID NO: 23 - protein, 9 aa, Artificial Sequence
  Synthetic CDR-L3 of anti-Ang2 antibody
QXDYSSPXT

SEQ ID NO: 24 - protein, 30 aa, Artificial Sequence
  Synthetic FR1 (N-terminal frame region of CDR-H1 of 10D6)
DVQLQESGPDLVKPSQSLSLTCTVTGYSIT

SEQ ID NO: 25 - protein, 30 aa, Artificial Sequence
  Synthetic FR1 (N-terminal frame region of CDR-H1 of VH-hu1)
QVQLQESGPGLVKPSETLSLTCAVSGYSIS

SEQ ID NO: 26 - protein, 30 aa, Artificial Sequence
  Synthetic FR1 (N-terminal frame region of CDR-H1 of VH-hu2)
QVQLQESGPGLVKPSETLSLTCAVSGYSIT

SEQ ID NO: 27 - protein, 30 aa, Artificial Sequence; identical to SEQ ID NO: 26
  Synthetic FR1 (N-terminal frame region of CDR-H1 of VH-hu5)

SEQ ID NO: 28 - protein, 30 aa, Artificial Sequence
  Synthetic FR1 (N-terminal frame region of CDR-H1 of VH-hu3)
EVQLVESGGGLVQPGGSLRLSCAASGYSIT

SEQ ID NO: 29 - protein, 14 aa, Artificial Sequence
  Synthetic FR2 (frame region between CDR-H1 and CDR-H2 of 10D6)
WIRQFPGNKLEWMG

SEQ ID NO: 30 - protein, 14 aa, Artificial Sequence
  Synthetic FR2 (frame region between CDR-H1 and CDR-H2 of VH-hu1)
WIRQPPGKGLEWIG

SEQ ID NO: 31 - protein, 14 aa, Artificial Sequence
  Synthetic FR2 (frame region between CDR-H1 and CDR-H2 of VH-hu2)
WIRQPPGKGLEWMG

SEQ ID NO: 32 - protein, 14 aa, Artificial Sequence
  Synthetic FR2 (frame region between CDR-H1 and CDR-H2 of VH-hu5)
WIRQPPGKGLEWIG

SEQ ID NO: 33 - protein, 14 aa, Artificial Sequence
  Synthetic FR2 (frame region between CDR-H1 and CDR-H2 of VH-hu3)
WVRQAPGKGLEWMG

SEQ ID NO: 34 - protein, 32 aa, Artificial Sequence
  Synthetic FR3 (frame region between CDR-H2 and CDR-H3 of 10D6)
RSSITRDTSKNQFFLQLNSVTTGDTATYYCAR

SEQ ID NO: 35 - protein, 32 aa, Artificial Sequence
  Synthetic FR3 (frame region between CDR-H2 and CDR-H3 of VH-hu1)
RVTISVDTSKNQFSLKLSSVTAADTAVYYCAR

SEQ ID NO: 36 - protein, 32 aa, Artificial Sequence
  Synthetic FR3 (frame region between CDR-H2 and CDR-H3 of VH-hu2)
RSTISRDTSKNQFSLKLSSVTAADTAVYYCAR

SEQ ID NO: 37 - protein, 32 aa, Artificial Sequence; identical to SEQ ID NO: 35
  Synthetic FR3 (frame region between CDR-H2 and CDR-H3 of VH-hu5)

SEQ ID NO: 38 - protein, 32 aa, Artificial Sequence
  Synthetic FR3 (frame region between CDR-H2 and CDR-H3 of VH-hu3)
RSTISRDTSKNTFYLQMNSLRAEDTAVYYCAR

SEQ ID NO: 39 - protein, 11 aa, Artificial Sequence
  Synthetic FR4 (C-terminal frame region of CDR-H3 of 10D6)
WGQGTSVTVSS

SEQ ID NO: 40 - protein, 11 aa, Artificial Sequence
  Synthetic FR4 (C-terminal frame region of CDR-H3 of VH-hu1)
WGQGTLVTVSS

SEQ ID NO: 41 - protein, 11 aa, Artificial Sequence
  Synthetic FR4 (C-terminal frame region of CDR-H3 of VH-hu2)
WGQGTLVTVSS

SEQ ID NO: 42 - protein, 11 aa, Artificial Sequence
  Synthetic FR4 (C-terminal frame region of CDR-H3 of VH-hu5)
WGQGTLVTVSS

SEQ ID NO: 43 - protein, 11 aa, Artificial Sequence
  Synthetic FR4 (C-terminal frame region of CDR-H3 of VH-hu3)
WGQGTLVTVSS

SEQ ID NO: 44 - protein, 23 aa, Artificial Sequence
  Synthetic FR1 (N-terminal frame region of CDR-L1 of 10D6)
SIVMTQTPKFLLVSAGDRVTITC

SEQ ID NO: 45 - protein, 23 aa, Artificial Sequence
  Synthetic FR1 (N-terminal frame region of CDR-L1 of VL-hu1)
DIQMTQSPSSLSASVGDRVTITC

SEQ ID NO: 46 - protein, 15 aa, Artificial Sequence
  Synthetic FR2 (frame region between CDR-L1 and CDR-L2 of 10D6)
WYQQKPGQSPKLLIY

SEQ ID NO: 47 - protein, 15 aa, Artificial Sequence
  Synthetic FR2 (frame region between CDR-L1 and CDR-L2 of VL-hu1)
WYQQKPGKAPKLLIY

SEQ ID NO: 48 - protein, 32 aa, Artificial Sequence
  Synthetic FR3 (frame region between CDR-L2 and CDR-L3 of 10D6)
GVPDRFTGSGYGTDFTFTISTVQAEDLAVYFC

SEQ ID NO: 49 - protein, 32 aa, Artificial Sequence
  Synthetic FR3 (frame region between CDR-L2 and CDR-L3 of VL-hu1)
GVPSRFSGSGSGTDFTLTISSLQPEDFATYYC

SEQ ID NO: 50 - protein, 10 aa, Artificial Sequence
  Synthetic FR4 (C-terminal frame region of CDR-L3 of 10D6)
FGGGTKLEIK

SEQ ID NO: 51 - protein, 10 aa, Artificial Sequence
  Synthetic FR4 (C-terminal frame region of CDR-L3 of VL-hu1)
FGQGTKVEIK

SEQ ID NO: 52 - protein, 118 aa, Artificial Sequence
  Synthetic Heavy chain variable region
QVQLQESGPGLVKPSETLSLTCAVSGYSITSDYAWNWIRQPPGKGLEWMGKISYSGKTDYNPSLKSRSTISRDTSKNQFSLKLSSVTAADTAVYYCARGNFEGAMDYWGQGTLVTVSS

SEQ ID NO: 53 - protein, 118 aa, Artificial Sequence
  Synthetic Heavy chain variable region
QVQLQESGPGLVKPSETLSLTCAVSGYSITSDYAWNWIRQPPGKGLEWMGKINYAGNTDYNPSLKSRSTISRDTSKNQFSLKLSSVTAADTAVYYCARGNFEGAMDYWGQGTLVTVSS

SEQ ID NO: 54 - protein, 118 aa, Artificial Sequence
  Synthetic Heavy chain variable region
EVQLVESGGGLVQPGGSLRLSCAASGYSITSDYAWNWVRQAPGKGLEWMGKISYSGKTDYNPSLKSRSTISRDTSKNTFYLQMNSLRAEDTAVYYCARGNFEGAMDYWGQGTLVTVSS

SEQ ID NO: 55 - protein, 118 aa, Artificial Sequence
  Synthetic Heavy chain variable region
EVQLVESGGGLVQPGGSLRLSCAASGYSITSDYAWNWVRQAPGKGLEWMGKINYAGNTDYNPSLKSRSTISRDTSKNTFYLQMNSLRAEDTAVYYCARGNFEGAMDYWGQGTLVTVSS

SEQ ID NO: 56 - protein, 118 aa, Artificial Sequence
  Synthetic Heavy chain variable region
QVQLQESGPGLVKPSETLSLTCAVSGYSITSDYAWNWIRQPPGKGLEWIGYINYSGNTDYNPSLKSRVTISVDTSKNQFSLKLSSVTAADTAVYYCARGNFEGAMDYWGQGTLVTVSS

SEQ ID NO: 57 - protein, 107 aa, Artificial Sequence
  Synthetic Light chain variable region
DIQMTQSPSSLSASVGDRVTITCKASQSVSNDVAWYQQKPGKAPKLLIYYASNRYPGVPSRFSGSGSGTDFTLTISSLQPEDFATYYCQQDYSSPWTFGQGTKLEIK

SEQ ID NO: 58 - protein, 107 aa, Artificial Sequence
  Synthetic Light chain variable region
DIQMTQSPSSLSASVGDRVTITCKASQSVSNDVHWYQQKPGKAPKLLIYYASNRYPGVPSRFSGSGSGTDFTLTISSLQPEDFATYYCQQDYSSPWTFGQGTKLEIK

SEQ ID NO: 59 - protein, 107 aa, Artificial Sequence
  Synthetic Light chain variable region
DIQMTQSPSSLSASVGDRVTITCKASQSVSNDVHWYQQKPGKAPKLLIYYASNRYPGVPSRFSGSGSGTDFTLTISSLQPEDFATYYCQHDYSSPFTFGQGTKLEIK

SEQ ID NO: 60 - protein, 107 aa, Artificial Sequence
  Synthetic Light chain variable region
DIQMTQSPSSLSASVGDRVTITCKASQFVSTDVHWYQQKPGKAPKLLIYYASNRYPGVPSRFSGSGSGTDFTLTISSLQPEDFATYYCQQDYSSPWTFGQGTKLEIK

SEQ ID NO: 61 - protein, 107 aa, Artificial Sequence
  Synthetic Light chain variable region
DIQMTQSPSSLSASVGDRVTITCKASQSVSNDVAWYQQKPGKAPKLLIYYASIPYPGVPSRFSGSGSGTDFTLTISSLQPEDFATYYCQQDYSSPWTFGQGTKLEIK

SEQ ID NO: 62 - protein, 107 aa, Artificial Sequence
  Synthetic Light chain variable region
DIQMTQSPSSLSASVGDRVTITCKASQSVSNDVHWYQQKPGKAPKLLIYYASIPYPGVPSRFSGSGSGTDFTLTISSLQPEDFATYYCQQDYSSPWTFGQGTKLEIK

SEQ ID NO: 63 - protein, 107 aa, Artificial Sequence
  Synthetic Light chain variable region
DIQMTQSPSSLSASVGDRVTITCKASQFVSTDVHWYQQKPGKAPKLLIYYASIPYPGVPSRFSGSGSGTDFTLTISSLQPEDFATYYCQQDYSSPWTFGQGTKLEIK

SEQ ID NO: 64 - DNA, 354 bp, Artificial Sequence
  Synthetic Coding sequence of heavy chain variable region(SEQ ID NO 52)
caggtgcaactgcaggagtcaggccccggcctggtaaaaccttctgaaacgctctcacttacctgtgccgttagtggatactctatcacttccgactacgcttggaattggattcggcagcctccaggcaaagggctggaatggatgggaaagatttcctattccggtaagactgactacaatcccagtctgaagagcaggtcaacaatctccagagacaccagcaagaatcagttttccctgaaattgtcctcggtgacagcagcggataccgcagtgtattattgcgcccgcggtaacttcgagggagctatggattactgggggcagggtactctcgtcactgtgagcagc

SEQ ID NO: 65 - DNA, 354 bp, Artificial Sequence
  Synthetic Coding sequence of heavy chain variable region(SEQ ID NO 53)
caggtgcaactgcaggagtcaggccccggcctggtaaaaccttctgaaacgctctcacttacctgtgccgttagtggatactctatcacttccgactacgcttggaattggattcggcagcctccaggcaaagggctggaatggatgggaaagattaactatgccggtaacactgactacaatcccagtctgaagagcaggtcaacaatctccagagacaccagcaagaatcagttttccctgaaattgtcctcggtgacagcagcggataccgcagtgtattattgcgcccgcggtaacttcgagggagctatggattactgggggcagggtactctcgtcactgtgagcagc

SEQ ID NO: 66 - DNA, 354 bp, Artificial Sequence
  Synthetic Coding sequence of heavy chain variable region(SEQ ID NO 54)
gaggttcagctggtcgaaagcggtgggggactcgtgcagccaggcggttctcttagattatcatgtgccgcatccgggtactccatcacctctgattatgcatggaactgggtcagacaagcccccggaaagggcctggagtggatggggaagatctcctattcagggaagacagattataatccttcgctgaaaagcagatcaacaattagtagagacacttctaaaaatactttttacctccagatgaacagtctgcgcgccgaagacaccgccgtgtactactgcgctaggggaaatttcgagggagctatggactattggggccagggcacgttggtaaccgtgagcagc

SEQ ID NO: 67 - DNA, 354 bp, Artificial Sequence
  Synthetic Coding sequence of heavy chain variable region(SEQ ID NO 55)
gaggttcaactggtagagtccgggggcggcctggtccagccaggaggaagcctgcggctctcttgtgccgccagcgggtatagtatcacttcagattatgcctggaattgggtccgccaggcccccgggaagggcttagagtggatgggtaaaattaattacgcaggcaacaccgactataatccttcactgaaatctagatccaccatctctagagatacaagtaagaacaccttttacttgcagatgaatagcctcagggctgaagacactgctgtgtactactgcgcaagaggaaacttcgaaggagcgatggattattggggccagggtacgcttgtgacagtgtcctct

SEQ ID NO: 68 - DNA, 354 bp, Artificial Sequence
  Synthetic Coding sequence of heavy chain variable region(SEQ ID NO 56)
caggtgcagctgcaggagtcgggcccaggactggtgaagccttcggagaccctgtccctcacctgcgctgtctctggttactccatcaccagtgattatgcctggaactggatccggcagcccccagggaaggggctggagtggattgggtacataaactacagtggtaacactgactacaacccatctctcaaaagtcgagtcaccatatcagtagacacgtccaagaaccagttctccctgaagctgagctctgtgaccgccgcagacacggccgtgtattactgtgcgagaggtaacttcgaaggtgctatggactactggggtcaaggaacgcttgtgacagtgtcctct

SEQ ID NO: 69 - DNA, 321 bp, Artificial Sequence
  Synthetic Coding sequence of light chain variable region(SEQ ID NO 57)
gacatccagatgacccagtctccatcctccctgtctgcatctgtaggagacagagtcaccatcacttgcaaggccagtcagagtgtgagtaatgatgtagcttggtatcagcagaaaccagggaaagcccctaagctcctgatctattatgcatccaatcgctaccctggggtcccatcaaggttcagtggcagtggatctgggacagatttcactctcaccatcagcagtctgcaacctgaagattttgcaacttactactgtcagcaggattatagctctccgtggacgttcggtggaggcaccaaggtggaaatcaaa

SEQ ID NO: 70 - DNA, 321 bp, Artificial Sequence
  Synthetic Coding sequence of light chain variable region(SEQ ID NO 58)
gacatccagatgacccagtctccatcctccctgtctgcatctgtaggagacagagtcaccatcacttgcaaggccagtcagagtgtgagtaatgatgtacattggtatcagcagaaaccagggaaagcccctaagctcctgatctattatgcatccaatcgctaccctggggtcccatcaaggttcagtggcagtggatctgggacagatttcactctcaccatcagcagtctgcaacctgaagattttgcaacttactactgtcagcaggattatagctctccgtggacgttcggtggaggcaccaaggtggaaatcaaa

SEQ ID NO: 71 - DNA, 321 bp, Artificial Sequence
  Synthetic Coding sequence of light chain variable region(SEQ ID NO 59)
gacatccagatgacccagtctccatcctccctgtctgcatctgtaggagacagagtcaccatcacttgcaaggccagtcagagtgtgagtaatgatgtacattggtatcagcagaaaccagggaaagcccctaagctcctgatctattatgcatccaatcgctaccctggggtcccatcaaggttcagtggcagtggatctgggacagatttcactctcaccatcagcagtctgcaacctgaagattttgcaacttactactgtcagcatgattatagctctccgttcacgttcggtggaggcaccaaggtggaaatcaaa

SEQ ID NO: 72 - DNA, 321 bp, Artificial Sequence
  Synthetic Coding sequence of light chain variable region(SEQ ID NO 60)
gacatccagatgacccagtctccatcctccctgtctgcatctgtaggagacagagtcaccatcacttgcaaggccagtcagttcgtgagtactgatgtacattggtatcagcagaaaccagggaaagcccctaagctcctgatctattatgcatccaatcgctaccctggggtcccatcaaggttcagtggcagtggatctgggacagatttcactctcaccatcagcagtctgcaacctgaagattttgcaacttactactgtcagcaggattatagctctccgtggacgttcggtggaggcaccaaggtggaaatcaaa

SEQ ID NO: 73 - DNA, 321 bp, Artificial Sequence
  Synthetic Coding sequence of light chain variable region(SEQ ID NO 61)
gacatccagatgacccagtctccatcctccctgtctgcatctgtaggagacagagtcaccatcacttgcaaggccagtcagagtgtgagtaatgatgtagcttggtatcagcagaaaccagggaaagcccctaagctcctgatctattatgcatccatcccataccctggggtcccatcaaggttcagtggcagtggatctgggacagatttcactctcaccatcagcagtctgcaacctgaagattttgcaacttactactgtcagcaggattatagctctccgtggacgttcggtggaggcaccaaggtggaaatcaaa

SEQ ID NO: 74 - DNA, 321 bp, Artificial Sequence
  Synthetic Coding sequence of light chain variable region(SEQ ID NO 62)
gacatccagatgacccagtctccatcctccctgtctgcatctgtaggagacagagtcaccatcacttgcaaggccagtcagagtgtgagtaatgatgtacattggtatcagcagaaaccagggaaagcccctaagctcctgatctattatgcatccatcccataccctggggtcccatcaaggttcagtggcagtggatctgggacagatttcactctcaccatcagcagtctgcaacctgaagattttgcaacttactactgtcagcaggattatagctctccgtggacgttcggtggaggcaccaaggtggaaatcaaa

SEQ ID NO: 75 - DNA, 321 bp, Artificial Sequence
  Synthetic Coding sequence of light chain variable region(SEQ ID NO 63)
gacatccagatgacccagtctccatcctccctgtctgcatctgtaggagacagagtcaccatcacttgcaaggccagtcagttcgtgagtactgatgtacattggtatcagcagaaaccagggaaagcccctaagctcctgatctattatgcatccatcccataccctggggtcccatcaaggttcagtggcagtggatctgggacagatttcactctcaccatcagcagtctgcaacctgaagattttgcaacttactactgtcagcaggattatagctctccgtggacgttcggtggaggcaccaaggtggaaatcaaa

SEQ ID NO: 76 - protein, 15 aa, Artificial Sequence
  Synthetic Linker
GGGGSGGGGSGGGGS

SEQ ID NO: 77 - protein, 118 aa, Artificial Sequence
  Synthetic 10D6-HU1
QVQLQESGPGLVKPSETLSLTCAVSGYSISSDYAWNWIRQPPGKGLEWIGYINYSGNTDYNPSLKSRVTISVDTSKNQFSLKLSSVTAADTAVYYCARGNFEGAMDYWGQGTLVTVSS

SEQ ID NO: 78 - protein, 118 aa, Artificial Sequence
  Synthetic 10D6-HU2
QVQLQESGPGLVKPSETLSLTCAVSGYSITSDYAWNWIRQPPGKGLEWMGYINYSGNTDYNPSLKSRSTISRDTSKNQFSLKLSSVTAADTAVYYCARGNFEGAMDYWGQGTLVTVSS

SEQ ID NO: 79 - protein, 118 aa, Artificial Sequence
  Synthetic 10D6-HU3
EVQLVESGGGLVQPGGSLRLSCAASGYSITSDYAWNWVRQAPGKGLEWMGYINYSGNTDYNPSLKSRSTISRDTSKNTFYLQMNSLRAEDTAVYYCARGNFEGAMDYWGQGTLVTVSS

SEQ ID NO: 80 - protein, 240 aa, Artificial Sequence
  Synthetic VH-linker-VL (scFv) of 10D6
DVQLQESGPDLVKPSQSLSLTCTVTGYSITSDYAWNWIRQFPGNKLEWMGYINYSGNTDYNPSLKSRSSITRDTSKNQFFLQLNSVTTGDTATYYCARGNFEGAMDYWGQGTLVTVSSGGGGSGGGGSGGGGSSIVMTQTPKFLLVSAGDRVTITCKASQSVSNDVAWYQQKPGQSPKLLIYYASNRYPGVPDRFTGSGYGTDFTFTISTVQAEDLAVYFCQQDYSSPWTFGGGTKLEIK

SEQ ID NO: 81 - DNA, 720 bp, Artificial Sequence
  Synthetic Coding sequence of VH-linker-VL (scFv) of 10D6
gatgtgcagcttcaggagtcgggacctgacctggtgaaaccttctcagtctctgtccctcacctgcactgtcactggctactcaatcaccagtgattatgcctggaactggatccggcagtttccaggaaacaaactggagtggatgggctacataaactacagtggtaacactgactacaacccatctctcaaaagtcgaagctctatcactcgagacacatccaagaaccagttcttcctgcagttgaattctgtgactactggggacacagccacatattactgtgcaagaggtaacttcgaaggtgctatggactactggggtcaaggaacctcagtcaccgtctcctcaggcggcggcggcagcggcggcggcggcagcggcggcggcggcagcagtattgtgatgacccagactcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctaccctggagtccctgatcgcttcactggcagtggatatgggacggatttcactttcaccatcagcactgtgcaggctgaagacctggcagtttatttctgtcagcaggattatagctctccgtggacgttcggtggaggcaccaagctggaaatcaaa

SEQ ID NO: 82 - protein, 240 aa, Artificial Sequence
  Synthetic VH-linker-VL (scFv) of10D6 opti-1
QVQLQESGPGLVKPSETLSLTCAVSGYSITSDYAWNWIRQPPGKGLEWMGKISYSGKTDYNPSLKSRSTISRDTSKNQFSLKLSSVTAADTAVYYCARGNFEGAMDYWGQGTLVTVSSGGGGSGGGGSGGGGSDIQMTQSPSSLSASVGDRVTITCKASQSVSNDVAWYQQKPGKAPKLLIYYASNRYPGVPSRFSGSGSGTDFTLTISSLQPEDFATYYCQQDYSSPWTFGQGTKLEIK

SEQ ID NO: 83 - DNA, 720 bp, Artificial Sequence
  Synthetic Coding sequence of VH-linker-VL (scFv) of 10D6 opti-1
caggtgcaactgcaggagtcaggccccggcctggtaaaaccttctgaaacgctctcacttacctgtgccgttagtggatactctatcacttccgactacgcttggaattggattcggcagcctccaggcaaagggctggaatggatgggaaagatttcctattccggtaagactgactacaatcccagtctgaagagcaggtcaacaatctccagagacaccagcaagaatcagttttccctgaaattgtcctcggtgacagcagcggataccgcagtgtattattgcgcccgcggtaacttcgagggagctatggattactgggggcagggtactctcgtcactgtgagcagcggcggcggcggcagcggcggcggcggcagcggcggcggcggcagcgacatccagatgacccagtctccatcctccctgtctgcatctgtaggagacagagtcaccatcacttgcaaggccagtcagagtgtgagtaatgatgtagcttggtatcagcagaaaccagggaaagcccctaagctcctgatctattatgcatccaatcgctaccctggggtcccatcaaggttcagtggcagtggatctgggacagatttcactctcaccatcagcagtctgcaacctgaagattttgcaacttactactgtcagcaggattatagctctccgtggacgttcggtggaggcaccaaggtggaaatcaaa

SEQ ID NO: 84 - protein, 118 aa, Artificial Sequence
  Synthetic Amino acid sequence of heavy chain variable region
DVQLQESGPGLVKPSQSLSLTCTVTGYSITSDYAWNWIRQFPGNKLEWMGYINYSGNTDYNPSLKSRSSITRDTSKNQFFLQLNSVTTGDTATYYCARGNFEGAMDYWGQGTLVTVSS

SEQ ID NO: 85 - protein, 30 aa, Artificial Sequence
  Synthetic FR1 (N-terminal frame region of CDR-H1 of 10D6)
DVQLQESGPGLVKPSQSLSLTCTVTGYSIT

SEQ ID NO: 86 - protein, 11 aa, Artificial Sequence
  Synthetic FR4 (C-terminal frame region of CDR-H3 of 10D6)
WGQGTLVTVSS

SEQ ID NO: 87 - protein, 107 aa, Artificial Sequence
  Synthetic Light chain variable region
DIQMTQSPSSLSASVGDRVTITCKASQSVSNDVAWYQQKPGKAPKLLIYYASNRYPGVPSRFSGSGSGTDFTLTISSLQPEDFATYYCGQDYASPWTFGQGTKLEIK

SEQ ID NO: 88 - DNA, 321 bp, Artificial Sequence
  Synthetic Nucleotide sequence coding VL (h10D6-OPTI-64)
gacatccagatgacccagtctccatcctccctgtctgcatctgtaggagacagagtcaccatcacttgcaaggccagtcagagtgtgagtaatgatgtagcttggtatcagcagaaaccagggaaagcccctaagctcctgatctattatgcatccaaccgataccctggggtcccatcaaggttcagtggcagtggatctgggacagatttcactctcaccatcagcagtctgcaacctgaagattttgcaacttactactgtggacaggattatgcctctccgtggacgttcggtggaggcaccaaggtggaaatcaaa

SEQ ID NO: 89 - protein, 107 aa, Artificial Sequence
  Synthetic Light chain variable region
DIQMTQSPSSLSASVGDRVTITCKASQSVSNDVHWYQQKPGKAPKLLIYYASNRYPGVPSRFSGSGSGTDFTLTISSLQPEDFATYYCGQDYSAPWTFGQGTKLEIK

SEQ ID NO: 90 - DNA, 321 bp, Artificial Sequence
  Synthetic Nucleotide sequence coding VL (h10D6-OPTI-66)
gacatccagatgacccagtctccatcctccctgtctgcatctgtaggagacagagtcaccatcacttgcaaggccagtcagagtgtgagtaatgatgtacactggtatcagcagaaaccagggaaagcccctaagctcctgatctattatgcatccaaccgataccctggggtcccatcaaggttcagtggcagtggatctgggacagatttcactctcaccatcagcagtctgcaacctgaagattttgcaacttactactgtggacaggattattctgccccgtggacgttcggtggaggcaccaaggtggaaatcaaa